(19) United States
(12) Patent Application Publication
(10) Pub. No.: US 2021/0238295 A1
(43) Pub. Date: Aug. 5, 2021

(54) COMPOSITIONS AND METHODS FOR TREATING ABDOMINAL AORTIC ANEURYSM

(71) Applicant: University of Virginia Patent Foundation, Charlottesville, VA (US)

(72) Inventor: Akshaya Kumar Meher, Charlottesville, VA (US)

(73) Assignee: University of Virginia Patent Foundation, Charlottesville, VA (US)

(21) Appl. No.: 17/050,231

(22) PCT Filed: Apr. 26, 2019
(86) PCT No.: PCT/US2019/029348

§ 371 Date: Oct. 23, 2020

Related U.S. Application Data
(60) Provisional application No. 62/662,849, filed on Apr. 26, 2018.

(51) Int. Cl. C07K 16/28; C07K 14/705

(52) U.S. Cl. CPC C07K 16/2875; A61K 2039/505; C07K 2317/34; C07K 14/70575

(57) ABSTRACT

Methods and compositions for inhibiting a soluble B cell activating factor (BAFF) biological activity are disclosed, as are methods for treating aortic aneurysm (AAA) are also disclosed. Exemplary compositions include a reagent, such as an antibody, that interacts with a BAFF polypeptide or BAFF gene product and a carrier, whereby inhibition of BAFF multimerization substantially without depletion of mature B cells is accomplished.

### RELATED APPLICATIONS

This application claims priority to and the benefit of U.S. Provisional Patent Application Ser. No. 62/662,849, filed on Apr. 26, 2018, the disclosure of which is incorporated herein by reference in its entirety.

### TECHNICAL FIELD

The presently disclosed subject matter relates to compositions and methods for treating abdominal aortic aneurysm.

### BACKGROUND

Abdominal aortic aneurysm (AAA) is characterized by enlargement of abdominal aorta, which can rupture, leading to death. With no nonsurgical treatment available, there is a great need to understand the mechanisms of AAA growth. B cells are present in human and experimental models of murine AAAs, and depletion of B cells protects mice from AAA. These results suggest a requirement for B cells in AAA growth. During the process of development, immature B cells migrate from bone marrow to spleen and differentiate into marginal zone and follicular B cells, which together constitute the B2 cell population, known to mediate humoral immunity. Moreover, during differentiation, B cells undergo metabolic reprogramming, e.g., antibody producing B cells are dependent on glycolysis and mitochondrial oxidative phosphorylation (OXPHOS). B cell differentiation is controlled by the soluble B cell activating factor (BAFF) which can bind to three surface receptors namely BAFF receptor (BR3), transmembrane activator, and CAML interactor (TACI), and B-cell maturation antigen (BCMA), B2 cells express BR3 and TACI receptors, and absence of BAFF leads to depletion of B2 cells. BR3 deficiency also depletes B2 cells, but TACI deficiency increases the number of B2 cells and immunoglobulin production. BR3 deficiency protects mice from atherosclerosis by lowering plasma IgG1 and IgG2a immunoglobulins. However, the role of BAFF in atherosclerosis and any other vascular disease is not clear.

Adding complexity to the BAFF-B cell interaction, soluble BAFF exists in two forms: as a 3mer, which binds only to BR3, or multimerizes to a highly active 60mer which binds to BR3, TACI, and BCMA. It is unknown whether the 3mer or the 60mer accounts for the pathogenic role of B2 cells. NF-kB signaling plays a role in B cell differentiation and BAFF activates both NF-kB1 and NF-kB2 signaling pathways. However, it is unknown if either BAFF 3mer or 60mer mediated NF-kB signaling provides a specific metabolic signature and pathogenic role to B2 cells. Treatment of WT B cells with the 60mer or injection of 60mer to BAFF−/− mice activates the B cells and induces IgG production.

There is a long felt need in the art for compositions and methods useful for treating AAA and for preventing the growth of AAA. The presently disclosed subject matter address these needs and other needs in the art, at least in part.

### SUMMARY

In some embodiments, the presently disclosed subject matter provides a composition for inhibiting a soluble B cell activating factor (BAFF) biological activity. In some embodiments, the composition comprises a reagent that interacts with a BAFF polypeptide or BAFF gene product and a carrier, whereby inhibition of BAFF multimerization substantially without depletion of mature B cells is accomplished. In some embodiments, the composition comprises an anti-BAFF antibody that binds to a BAFF polypeptide, a DE loop mimetic peptide, and/or a nucleic acid that binds to a BAFF gene product. In some embodiments, the anti-BAFF antibody binds to an epitope present within a DE loop of a BAFF polypeptide and/or comprising a subsequence thereof, wherein depletion of mature B cells is substantially avoided. In some embodiments, the BAFF biological activity is selected from the group consisting of BAFF multimerization, binding of BAFF to a cognate receptor, inducing signal transduction mediated by a cognate receptor, and modulating growth of an abdominal aortic aneurysm (AAA). In some embodiments, the BAFF multimerization comprises multimerization of a trimer form of BAFF to a 60mer form of BAFF In some embodiments, the cognate receptor is selected from the group consisting of BAFF receptor (BR3), CAML interactor (TACO, and B-cell maturation antigen (BCMA).

In some embodiments, provided is a pharmaceutical composition comprising a composition of the presently disclosed subject matter, optionally wherein the pharmaceutical composition is pharmaceutically acceptable for use in a human.

In some embodiments, provided is an anti-BAFF antibody formulation. In some embodiments, the anti-BAFF antibody formulation is prepared by immunizing a mammal with an antigen comprising an BAFF peptide or polypeptide. In some embodiments, the antigen comprises, consists essentially of, or consists of the amino acid sequence KVHVFGDELS (SEQ ID NO: 1). In some embodiments, the amino acid sequence KVHVFGDELS (SEQ ID NO: 1) is conjugated to a carrier. In some embodiments, the mammal is selected from the group consisting of a rabbit and a mouse. In some embodiments, the antigen comprises, consists essentially of, or consists of amino acid sequence KVHVFGDELSLVT (SEQ ID NO: 2) or amino acid sequence KVHVFGDELS (SEQ ID NO: 1), in some embodiments, optionally conjugated to keyhole limpet hemocyanin (KLH) via an N-terminal or a C-terminal cysteine addition. In some embodiments, the mammal is a mouse and the anti-BAFF antibody is a monoclonal antibody. In some embodiments, the formulation inhibits BAFF multimerization substantially without depletion of mature B cells.

In some embodiments, provided is a method for inhibiting a biological activity of a BAFF gene product. In some embodiments, the method comprises contacting the BAFF gene product with an effective amount of an inhibitor of BAFF, wherein the inhibitor comprises a reagent that interacts with a BAFF polypeptide or BAFF-encoding nucleic acid sequence, whereby inhibition of BAFF multimerization substantially without depletion of mature B cells is accomplished. In some embodiments, the inhibitor of BAFF is selected from the group consisting of an anti-BAFF antibody, a DE loop mimetic peptide, and an anti-BAFF inhibitory nucleic acid. In some embodiments, the anti-BAFF antibody binds to an epitope present within a DE loop of a BAFF polypeptide and/or comprising a subsequence thereof, to thereby inhibit BAFF multimerization, inhibit binding of BAFF to a cognate receptor, and/or inhibit signal transduction mediated by a cognate receptor. In some embodiments, the biological activity of the BAFF gene product is associated with growth of an abdominal aortic aneurysm (AAA).

In some embodiments, provided is a method for inhibiting growth of an abdominal aortic aneurysm (AAA). In some embodiments, the method comprises administering to a subject in need thereof an effective amount of a composition for inhibiting a soluble B cell activating factor (BAFF) biological activity. In some embodiments, the composition for inhibiting a soluble B cell activating factor (BAFF) biological activity comprises a reagent that interacts with a BAFF polypeptide or BAFF-encoding nucleic acid sequence and a carrier. In some embodiments, the composition inhibits BAFF multimerization substantially without depletion of mature B cells. In some embodiments, the composition comprises an anti-BAFF antibody that binds to a BAFF polypeptide, a DE loop mimetic peptide, and/or a nucleic acid that binds to a BAFF gene product. In some embodiments, the anti-BAFF antibody binds to an epitope present within a DE loop of a BAFF polypeptide and/or comprising a subsequence thereof. In some embodiments, the BAFF biological activity is selected from the group consisting of BAFF multimerization, binding of BAFF to a cognate receptor, inducing signal transduction mediated by a cognate receptor, and modulating growth of an abdominal aortic aneurysm (AAA), In some embodiments, BAFF multimerization comprises multimerization of a trimer form of BAFF to a 60mer form of BAFF. In some embodiments, the cognate receptor is selected from the group consisting of BAFF receptor (BR3), CAML interactor (TACI), and B-cell maturation antigen (BCMA). In some embodiments, the composition comprises a pharmaceutically acceptable carrier, in some embodiments, optionally wherein the carrier is pharmaceutically acceptable for use in a human.

In some embodiments, provided is a method of treating a B cell-related condition. In some embodiments, the method comprises administering to a subject in need thereof an effective amount of a composition of the presently disclosed subject matter. In some embodiments, the B cell-related condition comprises a condition selected from the group consisting of an abdominal aortic aneurysm (AAA), a cardiovascular disease, lupus, type 1 diabetes, type 2 diabetes, and a B-cell related cancer.

Accordingly, it is an object of the presently disclosed subject matter to provide compositions and methods for treating AAA and for preventing the growth of AAA. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description of the presently disclosed subject matter and non-limiting Figures and Examples.

### BRIEF DESCRIPTION OF THE DRAWINGS

FIG. 1 shows that BAFF receptors are expressed in human and mouse AAAs. Immunohistochemistry of aortic cross-sections from human and mouse AAAs show expression of BAFF receptors and presence of B cells (CD79a for human and CD20 for mouse). Arrows indicate co-localized staining of B cell and BAFF receptors, and “*” indicates lumen.

FIGS. 2A-2D show pharmacological inhibition of BAFF or blocking BR3 receptor attenuates AAA formation in mice. FIGS. 2A and 2B, in a prevention strategy, WT mice were intravenously injected with 1 or 2 mg/kg of anti-BAFFAb, 2 mg/kg anti-BR3 Ab, or a control Ab once in 14 days. After 14 days of the first Ab treatment, AAA was induced via topical elastase model. Increase in aorta diameter was determined after 14 days of AAA induction.

FIG. 2C, in a treatment strategy, AAA was induced in three groups of WT mice via topical elastase model and 7 days after the AAA induction, mice were treated intravenously with a control Ab, anti-BAFF, or anti-BR3 Ab (n=6/group). Seven days after the Ab treatment, AAA size was determined. As a control, AAA size was determined in 7 days after AAA induction (group Day 7, n=6). FIG. 2D, Serial aortic cross sections from the treatment group were stained to examine degradation of elastin layer (VVG), smooth muscle cell layer (alpha smooth muscle actin), expression of MMP9, presence of neutrophils, macrophages, B cells, immunoglobulin and IgM. As a control, an aortic section was stained without the primary(1°) Ab. Hematoxylin (blue/purple) staining was used to identify the nuclei. Values are expressed as means SEM. *, p<0.05; **, p<0.01; and ***, p<0.001 by Student's t-test.

FIGS. 3A and 3B show that anti-BAFF or anti-BR3 treatment depletes plasma IgG2 level. FIG. 3A, Plasma immunoglobulin levels were quantified using ELISA from the blood collected from the prevention and treatment groups (n=4-5). FIG. 3B, In a control experiment to examine the effect of injection of control Ab on the levels of plasma immunoglobulins, WI mice were injected with 2 mg/kg of the murine monoclonal IgG1 and plasma immunoglobulins were quantified before the injection, and 7, and 14 days after the injection (n=4). Injection of monoclonal IgG1 significantly increased the level of plasma IgG1 suggesting further validation of the significant differences in IgG1 levels in FIG. 3A. However, blocking BAFF or the BR3 lowered IgG2 level as found in BAFF or BR3 KO mice. Values are expressed as means+SEM. *, p<0.05; **, p<0.01; and ***, p<0.001 by Student's t-test.

FIGS. 4A and 4B show that anti-BAFF or anti-BR3 treatment depletes mature B cell subpopulations. FIG. 4A, representative flow cytometry plots showing depletion of T2, FO, MZP and MZ, but not T2 B cells in spleen after the anti-BAFF treatment. FIG. 4b quantification of B cell sub-types in spleen in the prevention and treatment groups (n=4-6). Values are expressed as means+SEM. *, p<0.05; ** p<0.01; and ***, p<0.001 by Student's t-test

FIGS. 5A-5F show that BAFF 60mer induces genes required for B cell activation, which are partly suppressed by blocking BR3. FIG. 5A, schematic presentation of interaction of BAFF 3mer and 60mer with BAFF receptors. FIG. 5B, clustered heat map of 963 genes that were differentially expressed in isolated total B cells after 4 hours of treatment with 100 ng/ml of BAFF3mer, 60mer or left untreated (UT) in presence of the control Ab or anti-BR3 Ab. Expression of genes (relative to UT) involved in B cell activation.

FIG. 5C, NF-kB signaling. FIG. 5D, metabolism. FIG. 5E, scale, log 2 (fold change). FIG. 5F, PANTHER analysis of pathways regulated by BAFF 3mer and 60mer in presence of the control or anti-BR3 Ab.

FIGS. 6A-6D show that anti-BR3 Ab inhibits BAFF 60 mer-induced NF-kB1 signaling but does not affect expression of B cell activation markers.

FIG. 6A, Western blot analysis of NF-kB1 and -kB2 signaling from whole cell lysate of B cells isolated from mouse spleen treated with BAFF 3mer or 60mer for 3 hours or 24 hours, or left untreated (UT) (n=3/group). FIG. 6B, flow cytometry quantification of B cell activation markers CD23 and MHCII after treating B cells with BAFF 3mer or 60mer for 24 hours, or left untreated (UT). To determine the effect of NF-kB signaling on the expression of CD23 and MHCII, the cells were pre-treated for 1 hour with BMS, SNSO, and SN52 (inhibits NF-kB signaling, nuclear translocation of p50, and p52, respectively) or with vehicle (PBS) followed by treatment with BAFF 3mer, 60mer or left untreated (n=4/group). FIG. 6C, Western blot analysis of NF-kB1 and -kB2 signaling from whole cell lysate of B cells treated with BAFF 3mer or 60mer, or left untreated (UT) for 3 hours in presence of the control Ab or anti-BR3 Ab (n=3/group). FIG. 6D, Flow cytometry quantification of CD23 and MHCII after treating B cells with BAFF 3mer or 60mer for 24 hours, or left untreated (UT) in presence of the control Ab or anti-BR3 Ab (n=4/group). MFI indicates mean fluorescence intensity and values are expressed as means+SD, and “*” “**” and “***” indicate p<0.05, 0.01 and 0.001, respectively.

FIGS. 7A-7E show that anti-BR3 Ab inhibits BAFF 60mer-induced metabolic reprogramming in Bcells. FIG. 7A, Cellular respiration of B cells was determined after treatment with various concentrations of with BAFF 3mer or 60mer for 24 hours, 1 μg/ml LPS, 10 μg/ml anti-C©40 Ab or left untreated (UT) by a mitochondrial stress test and a glycolytic stress test on Seahorse XF24 Analyzer. FIG. 7B, Flow cytometry quantification of CD23 and MHCII after treatment with BAFF 3mer or 60mer for 24 hours or left untreated (UT) for 24 hours. Before the treatment with BAFF, B cells were pre-treated with AA±Rot, 2-DG, or TOFA for 1 hours (n=4/group). FIG. 7C, Maximal and reserve OCR of B cells treated with BAFF 3mer and 60mer in presence of the control Ab or anti-BR3 Ab. FIG. 7D, Maximal and reserve ECAR of B cells treated with BAFF 3mer and 60mer in presence of the control Ab or anti-BR3 Ab. FIG. 7E, The effect of SN50 and SN52 on BAFF 60mer-induced OCR and ECAR. OCR, Oxygen consumption rate; ECAR, extracellular acidification rate; values are expressed as means+SE, n=4-5/group and “*”, “**” and “***” indicate p<0.05, 0.01 and 0.001, respectively.

FIGS. 8A and 8B show that the anti-DE3 Ab binds to DE loop of BAFF. FIG. 8A, ribbon structure of BAFF generated using Swiss-PDB viewer 4.1.0 showing the DE loop of one BAFF involved in interacting with two other BAFF molecules to form the 60mer. FIG. 8B, A 96-well ELISA plate was plated with 10 ng of BAFF 60mer for overnight. Subsequently, the plate was washed, blocked, and various dilutions of anti-DE3 Ab, anti-DE3 Ab+DE peptide and a control IgG were added. The plate was washed to remove unbound Abs and bound Abs were detected using HRP— conjugated secondary Abs and TBM substrate. A stop solution was added before quantifying the color development at OD 450 nm. Values are expressed as means±SD and n=3 per group, Significant difference between the curves was calculated by 2-way ANNOVA. * and *** represent p<0.05 and 0.001, respectively.

FIG. 9 is a series of plots and graphs showing that anti-DE3 Ab treatment does not affect the population of mature B cells. As indicated, 8 weeks old male C57BL/6 mice were injected with 100 ug of anti-DE3 Ab or a control Ab (n=3/group). After collecting the whole spleen, single cells suspension was prepared, RBCs were lysed and the cells were stained for live/dead stain before staining with fluorescent-conjugated B cell surface markers, After staining, the cells were fixed, counting beads were added and run on the flow cytometer LSR Fortessa. Values are expressed as means+SD. * and *** represent p<0.05 and 0.001, respectively by a Student's t-test.

FIG. 10 is a series of graphs showing that anti-DE3 Ab suppresses the expression level of B cell activation markers. Median fluorescent intensity of CD21, CD23 and MHCII expressed on FO B cells of spleen and blood (from the experiment described in FIG. 9). Values are expressed as means+SD. *, ** and *** represent p<0.05, 0.01 and 0.001, respectively by a Student's t-test.

FIG. 11 shows that neutrophils localize close to B cells and plasma cells are present in AAAs of WT mice. Left panels: Co-localization of neutrophils (LyB.2, brown) and B cell (CD20, black) in mouse AAA. Right panels: Presence of immunoglobulins (brown) and plasma cells (CD138, black) in mouse AAA.

### DETAILED DESCRIPTION

While the role of BAFF in atherosclerosis and any other vascular disease is not clear, the presently disclosed subject matter relates in some embodiments to the demonstration that an anti-BAFF antibody treatment depletes the B2 cells, lowers plasma IgG1 and IgG2a levels, and suppresses AAA growth in mice. These results suggest that BAFF promotes AAA growth.

In accordance with aspects of the presently disclosed subject matter, BAFF 3mer induces NF-kB2, whereas, 60mer induces both NF-kB1 and NF-kB2 signaling. The 3mer marginally increased NF-kB2 signaling and expression of B cell survival genes, whereas the 60mer significantly increased NF-kB1 and NF-kB2 signaling, B cell survival and activation genes, OXPHOS and glycolysis, suggesting differentiation to plasma cells. Other aspects of the presently disclosed subject matter relate to that BAFF multimerization regulates the immune and metabolic phenotype of B cells by binding to BAFF receptors and modulate AAA growth.

Without wishing to be bound by any particular theory, it is hypothesized herein that the BAFF 60mer binding to B2 cells induces NF-kB signaling and metabolism leading to increased B2 cell activation, IgG production, and AAA growth. In sterile inflammation, such as cardiovascular diseases, B2 B cells are considered harmful via secretion of pathogenic antibodies. B cell differentiation is controlled by the soluble B cell activating factor (BAFF) which can bind to three surface receptors, namely BAFF receptor (BR3), transmembrane activator and CAVIL interactor (TACI), and B-cell maturation antigen (BCMA). Soluble BAFF exists in two forms: as a 3mer, which binds only to BR3, or multimerizes to a highly active 60mer which binds to BR3, TACI and BCMA ((Liu et al., Cell. 2002; 108(3):383-94), (Bassen et al. Blood. 2008; 111(3):1004-12)). BAFF 60mer formation requires the solvent accessible DE loop. Mutation in the Histidine 218 to Alanine (H218A) of DE loop in human BAFF inhibits oligomerization of the 3mer to the 60mer. The binding affinity of the 3mer to the BR3 receptor is similar to that of 60mer, however, the 60mer is more active than 3mer in inducing proliferation of activated B cells (Ca hero et al. Biochemistry. 2006; 45(7):2006-13)). Injection of 40 μg/mouse (i.p. daily) of H218A BAFF (3mer) to BAFF^{-/-} mice restores peripheral B cell populations and antibody responses (Bossen et al. Eur J immunol. 2011; 41(3):787-97)), However, injection of the 60mer not only increases the level of CD23 expression on B cells, but also increases the number of B2 cells. Based on these results but without wishing to be bound by any particular theory of operation, it is hypothesized that BAFF 60mer binding activates B cells and promotes B cell mediated pathogenesis in multiple diseases. Therefore, reagents inhibiting 60mer formation can be beneficial for many diseases.

The present data demonstrates that blocking BR3 via anti-BR3 IgG1 aggravate aortic aneurysm growth in mice, which suggests that, 60mer activity promotes AAA growth. Therefore, the presently disclosed subject matter relates at least in part to methods and compositions for inhibition of 60mer formation and suppression of AAA growth in subjects. Aspects of the presently disclosed subject matter relate to BAFF multimerization regulates the immune and metabolic phenotype of B cells by binding to BAFF receptors and modulate AAA growth and to 60mer promotes AAA growth by inducing differentiation of naïve B cells to antibody secreting plasma cells.

The presently disclosed subject matter adds to the understanding of regulation of B cell function by BAFF multimerization and role of B cells in AAA. Novel reagents and methods are provided, which provide for development of treatment strategies for AAA and other B cell-related diseases such as cardiovascular disease, type 1 and type 2 diabetes and lupus.

Various aspects and embodiments of the presently disclosed subject matter are described in further detail below.

### I. General Considerations:

Abdominal aortic aneurysm (AAA) is characterized by enlargement of abdominal aorta. Except for invasive surgical interventions, no other treatment strategy is available to inhibit the growth or rupture of established AAA. The presently disclosed subject matter involves the study of immunopathogenesis of AAA to develop a non-surgical treatment strategy. During AAA formation, increased secretion of matrix metalloproteinases degrades aortic fibers and weakens the aortic wall. Neutrophils and B cells infiltrate aorta during AAA formation and depletion of either of the cell types attenuates inflammation, retains aortic structure, and protects mice from AAA. This would appear to implicate a role for neutrophil-B cell inflammatory crosstalk during AAA formation. Aspects of the presently disclosed subject matter relate to approaches to inhibit this crosstalk, which would attenuate inflammation and AAA formation in a subject without depletion of B cells or neutrophils.

Neutrophils secrete B cell activating factor (BAFF), which can regulate B cell function by binding to BAFF receptors. While the role of BAFF in vascular diseases is unknown, the presently disclosed subject matter demonstrates that anti-BAFF antibody treatment depletes 82 B cells, attenuates AAA formation, retains aortic structure and decreases plasma level of only two of the antibodies IgG1 and IgG2, which are known to be the pathogenic in atherosclerosis. IgGs can activate Fcγ receptors (FcγRs) and contribute to vascular pathogenesis. As described elsewhere herein, BAFF deficiency did not affect neutrophil infiltration and FcγRIII expression, but decreased IgG deposition in aortic wall. These results suggest a role for neutrophil secreted BAFF in B2 cell activation, pathogenic IgG production, and AAA formation.

BAFF is secreted as a 3mer, which can bind to the BAFF receptor BR3. Interestingly, the 3mer can associate (via its DE loop region) to a 60mer which can bind to three of the BAFF receptors BR3, TACI and BCMA. Antibody producing B cells are differentiated from activated B2 cells, which express the receptors 8R3 and TACI. Cell signaling, RNA sequencing, and metabolism studies disclosed elsewhere herein suggest that the 60mer, but not the 3mer, activates B cells and promotes metabolic reprogramming, which are attenuated by inhibition of NF-kB signaling or by blocking BR3 with an anti-BR3 antibody. In accordance with the presently disclosed subject matter, a novel polyclonal antibody against the DE loop of BAFF (anti-DE3 Ab) does not deplete B2 cells but suppresses B cell activation in mice Taking these findings together, and while it is not desired to be bound by any particular theory of operation, it is believed that the neutrophil-secreted BAFF form 60mer which activates B2 cells to produce pathogenic IgGs, damages the aorta and promotes AAA formation and that that inhibition of 60mer formation suppresses neutrophil-mediated B2 cell activation and attenuates AAA formation.

B2 B cells are proinflammatory in vascular diseases. B cells are uniquely placed in modulating vascular diseases because of their effector function, i.e. production of antibodies. Broadly, B cells are grouped in to B1 and B2 cell. B1 cells protect mice from atherosclerosis by secreting natural IgMs. It is thought that B2 cells are pro-inflammatory by secreting pathogenic antibodies that bind to the vascular wall and promotes atherosclerosis. More than 80% of B cells in spleen and blood are naïve B2 cells. Upon activation, naïve B2 cells proliferate and differentiate in to antibody producing plasma cell B cells. Differentiation and proliferation of B2 cell is highly dependent on B cell activating factor (BAFF) and the BAFF receptor BR3. Whereas, the role of BAFF in vascular disease is unknown, genetic or pharmacological deficiency of BR3 leads to >85% reduction in B2 cells number (but not in B1 cells), significant decrease in IgG1 and IgG2a levels in plasma, decrease in deposition of IgGs in aorta, decrease in inflammation, and protection of mice from atherosclerosis. Thus, understanding BAFF signaling will help in targeting B2 cells and lower the levels of pathogenic IgGs.

### II. Definitions

In describing and claiming the presently disclosed subject matter, the following terminology will be used in accordance with the definitions set forth below.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about,”

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As used herein, the term “adjuvant” refers to a substance that elicits an enhanced immune response when used in combination with a specific antigen.

As use herein, the terms “administration of” and or “administering” a compound should be understood to mean providing a compound of the presently disclosed subject matter or a prodrug of a compound of the presently disclosed subject matter to a subject in need of treatment.

As used herein, the term “aerosol” refers to suspension in the air. In particular, aerosol refers to the particlization or atomization of a formulation of the presently disclosed subject matter and its suspension in the air.

The term “alterations in peptide structure” as used herein refers to changes including, but not limited to, changes in sequence, and post-translational modification.

As used herein, “alleviating a disease or disorder symptom,” means reducing the severity of the symptom or the frequency with which such a symptom is experienced by a patient, or both.

As used herein, amino acids are represented by the full name thereof, by the three-letter code corresponding thereto, or by the one-letter code corresponding thereto, as indicated in the following table:

TABLE 1
Amino Acids and Functionally
Equivalent Codons

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The term “amino acid” is used interchangeably with “amino acid residue,” and may refer to a free amino acid and to an amino acid residue of a peptide. It will be apparent from the context in which the term is used whether it refers to a free amino acid or a residue of a peptide. Amino acids can be classified into seven groups on the basis of the side chain: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)2, as well as single chain antibodies and humanized antibodies.

The term “antibody” refers to polyclonal and monoclonal antibodies and derivatives thereof (including chimeric, synthesized, humanized and human antibodies), including an entire immunoglobulin or antibody or any functional fragment of an immunoglobulin molecule which binds to the target antigen and or combinations thereof. Examples of such functional entities include complete antibody molecules, antibody fragments, such as Fv, single chain Fv, complementarity determining regions (CDRs), VL (light chain variable region), VH (heavy chain variable region), Fab, F(ab′)2 and any combination of those or any other functional portion of an immunoglobulin peptide capable of binding to target antigen.

Antibodies exist, e.g., as intact immunoglobulins or as a number of well characterized fragments produced by digestion with various peptidases. Thus, for example, pepsin digests an antibody below the disulfide linkages in the hinge region to produce F(ab′)2 a dimer of Fab which itself is a light chain joined to VH —CH1 by a disulfide bond. The F(ab′)2 may be reduced under mild conditions to break the disulfide linkage in the hinge region, thereby converting the F(ab′)2 dialer into an Fab1 monomer. The Fab1 monomer is essentially a Fab with part of the hinge region (see, FUNDAMENTAL IMMUNOLOGY, 3RD ED., W. E. Paul, ed, Raven Press, N.Y. (1993)). While various antibody fragments are defined in terms of the digestion of an intact antibody, one of skill will appreciate that such fragments may be synthesized de novo either chemically or by utilizing recombinant DNA methodology, Thus, the term antibody, as used herein, also includes antibody fragments either produced by the modification of whole antibodies or those synthesized de novo using recombinant DNA methodologies.

An “antibody heavy chain.” as used herein, refers to the larger of the two types of polypeptide chains present in all antibody molecules.

An “antibody light chain,” as used herein, refers to the smaller of the two types of polypeptide chains present in all antibody molecules.

The term “single chain antibody” refers to an antibody wherein the genetic information encoding the functional fragments of the antibody are located in a single contiguous length of DNA. For a thorough description of single chain antibodies, see Sire, et al., Science 242:423 (1988) and Huston, et al., Proc. Nat'l Acad. Sci. USA 85:5879 (1988).

The term “humanized” refers to an antibody wherein the constant regions have at least about 80% or greater homology to human immunoglobulin. Additionally, some of the nonhuman, such as murine, variable region amino acid residues can be modified to contain amino acid residues of human origin.

Humanized antibodies have been referred to as “reshaped” antibodies. Manipulation of the complementarity-determining regions (CDR) is a way of achieving humanized antibodies, See, for example, Jones, et al., Nature 321:522 (1988) and Riechmann, et al., Nature 332:323 (1988), both of which are incorporated by reference herein. For a review article concerning humanized antibodies, see Winter & Milstein, Nature 349:293 (1991), incorporated by reference herein.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded. DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

Antisense technology has been demonstrated to be an effective method of modifying the expression levels of gene products (see, for example, U.S. Pat. Nos. 8,765,703, 8,946,183, and U.S. Patent Publication No. 2015/0376615, which are incorporated herein by reference in their entirety). Antisense technology works by interfering with known steps in the normal processing of mRNA. Briefly, RNA molecules are transcribed from genomic DNA in the nucleus of the cell. These newly synthesized mRNA molecules, called primary mRNA or pre-mRNA, must be processed prior to transport to the cytoplasm for translation into protein at the ribosome, Such processing includes the addition of a 5′ methylated cap and the addition of a poly(A) tail 10 to the 3′end of the mRNA.

In one application of antisense technology, an antisense oligonucleotide (AON) binds to a mRNA molecule transcribed from a gene of interest and inactivates (“turns off”) the mRNA by increasing its degradation or by preventing translation or translocation of the mRNA by steric hindrance. The end result is that expression of the corresponding gene (i.e., final production of the protein encoded by the corresponding gene) is prevented. Alternatively, antisense technology can be used to affect splicing of a gene transcript. In this application, the antisense oligonucleotide binds to a pre-spliced RNA molecule (pre-messenger RNA or pre-mRNA) and re-directs the cellular splicing apparatus, thereby resulting in modification of the exon content of the spliced mRNA molecule. Thus, the overall sequence of a protein encoded by the modified mRNA differs from a protein translated from mRNA, the splicing of which was not altered (i.e., the full length, wild-type protein). The protein that is translated from the altered mRNA may be truncated and/or it may be missing critical sequences required for proper function, Typically, the compounds used to affect splicing are, or contain, oligonucleotides having a base sequence complementary to the mRNA being targeted. Such oligonucleotides are referred to herein as “antisense oligonucleotides” (AONs).

An “aptamer” is a compound that is selected in vitro to bind preferentially to another compound (for example, the identified proteins herein). Often, aptamers are nucleic acids or peptides because random sequences can be readily generated from nucleotides or amino acids (both naturally occurring or synthetically made) in large numbers but of course they need not be limited to these.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner,” as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the polypeptides encompasses natural or synthetic portions of the full-length protein that are capable of specific binding to their natural ligand or of performing the function of the protein.

The term “biological sample,” as used herein, refers to samples obtained from a subject, including, but not limited to, sputum, mucus, phlegm, tissues, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. One of skill in the art will understand the type of sample needed.

A “biomarker” or “marker” is a specific biochemical in the body which has a particular molecular feature that makes it useful for measuring the progress of disease or the effects of treatment, or for measuring a process of interest.

The term “cancer”, as used herein, is defined as proliferation of cells whose unique trait (loss of normal controls) results in unregulated growth, lack of differentiation, local tissue invasion, and metastasis. Examples include but are not limited to, B cell cancers such as lymphomas.

As used herein, the term “carrier molecule” refers o any molecule that is chemically conjugated to a molecule of interest.

The term “cell surface protein” means a protein found where at least part of the protein is exposed at the outer aspect of the cell membrane. Examples include growth factor receptors.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates, or carboxylic acids, on the antigen and the carrier molecule, Chemical conjugation also includes non-covalent linkage between the antigen and the carrier molecule.

A “coding region” of a gene includes the nucleotide residues of the coding strand of the gene and the nucleotides of the non-coding strand of the gene which are homologous with or complementary to, respectively, the coding region of an mRNA molecule which is produced by transcription of the gene.

The term “competitive sequence” refers to a peptide or a modification, fragment, derivative, or homolog thereof that competes with another peptide for its cognate binding site.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., NT and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion. A “compound,” as used herein, refers to any type of substance or agent that is commonly considered a drug, or a candidate for use as a drug, as well as combinations and mixtures of the above.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media, Information may be transferred between a system of interest and a medium, between computers, or between computers and the computer-readable medium for storage or access of stored information. Such transmission can be electrical, or by other available methods, such as IR links, wireless connections, etc.

As used herein, the term “conservative amino acid substitution” is defined herein as an amino acid exchange within one of the following five groups:
Small aliphatic, nonpolar or slightly polar residues: Ala, Ser, Thr, Pro, Gly;
Polar, negatively charged residues and their amides; Asp, Asn, Glu, Gln;
Polar, positively charged residues: His, Arg, Lys;
Large, aliphatic, nonpolar residues: Met Leu, He, Val, Cys
Large, aromatic residues: Phe, Tyr, Tip

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

As used herein, a “derivative” of a compound refers to a chemical compound that may be produced from another compound of similar structure in one or more steps, as in replacement of H by an alkyl, acyl, or amino group.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A “condition” encompasses both diseases and disorders.

As used herein, the term “domain” refers to a part of a molecule or structure that shares common physicochemical features, such as, but not limited to, hydrophobic, polar, globular and helical domains or properties such as ligand binding, signal transduction, cell penetration and the like. Specific examples of binding domains include, but are not limited to, DNA binding domains and ATP binding domains.

As used herein, an “effective amount” or “therapeutically effective amount” means an amount sufficient to produce a selected effect, such as alleviating symptoms of a condition, including a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein, the term “effector domain” refers to a domain capable of directly interacting with an effector molecule, chemical, or structure in the cytoplasm, which is capable of regulating a biochemical pathway.

The term “elixir,” as used herein, refers in general to a clear, sweetened, alcohol-containing, usually hydroalcoholic liquid containing flavoring substances and sometimes active medicinal agents.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system, Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly five amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “subsequence,” “fragment” or “segment” is a portion of an amino acid sequence, comprising at least one amino acid, or a portion of a nucleic acid sequence comprising at least one nucleotide. The terms “subsequence,” “fragment” and “segment” are used interchangeably herein.

As used herein, the term “fragment,” as applied to a protein or peptide, can ordinarily be at least about 3-15 amino acids in length, at least about 15-25 amino acids, at least about 25-50 amino acids in length, at least about 50-75 amino acids in length, at least about 75-100 amino acids in length, and greater than 100 amino acids in length.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci, USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402), Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, the term “inhaler” refers both to devices for nasal and pulmonary administration of a drug, e.g., in solution, powder and the like. For example, the term “inhaler” is intended to encompass a propellant driven inhaler, such as is used to administer antihistamine for acute asthma attacks, and plastic spray bottles, such as are used to administer decongestants.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block,”

The term “inhibit a complex,” as used herein, refers to inhibiting the formation of a complex or interaction of two or more proteins, as well as inhibiting the function or activity of the complex. The term also encompasses disrupting a formed complex.

However, the term does not imply that each and every one of these functions must be inhibited at the same time.

The term “inhibit a protein,” as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

As used herein “injecting or applying” includes administration of a compound of the presently disclosed subject matter by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means. Compounds or agents of the presently disclosed subject matter can be administered to a subject by these approaches when appropriate.

As used herein, the term “invasive,” or “metastasis” as used herein, refers to any migration of cells, especially to invasive cancer cells or tumor cells. The term applies to normally invasive cells such as wound-healing fibroblasts and also to cells that migrate abnormally. Although the term is not to be limited by any mechanistic rationale, such cells are thought to migrate by defeating the body's means for keeping them sufficiently “in place” to function normally. Such cells are “invasive” if they migrate abnormally within a tissue or tumor, or escape the tissue, or invade other tissues.

As used herein, the terms “interact” and grammatical variations thereof, is meant to refer to any type of interaction that might occur between two chemical entities. Thus, the term “interact” refers to the following representative, non-limiting list: covalent bonding, ionic bonding, van der Waal interactions, hydrophobic interactions, binding between an antibody and an antigen, binding between a ligand and a receptor, binding between complementary nucleic acid sequences, interactions between peptides, interactions between polypeptides, interactions between a peptide and a polypeptide, small molecule interactions, and any other interaction as might be apparent to one of ordinary skill in the art upon review of the instant disclosure.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

A “ligand” is a compound that specifically binds to a target receptor.

A “receptor” is a compound that specifically binds to a ligand.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Wass interactions, e.g., a nucleic acid molecule that hybridizes to one complementary sequence at the 5′ end and to another complementary sequence at the 3′ end, thus joining two non-complementary sequences.

As used herein, the term “malignant” refers to having the properties of anaplasia, penetrance, such as into nearby areas or the vasculature, and metastasis.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The term “nasal administration” in all its grammatical forms refers to administration of at least one compound of the presently disclosed subject matter through the nasal mucous membrane to the bloodstream for systemic delivery of at least one compound of the presently disclosed subject matter. The advantages of nasal administration for delivery are that it does not require injection using a syringe and needle, it avoids necrosis that can accompany intramuscular administration of drugs, and trans-mucosal administration of a drug is highly amenable to self-administration.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the presently disclosed subject matter. By “nucleic 30 acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired; whether obtained by genomic or synthetic methods.

Unless otherwise specified; a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “peptide” typically refers to short polypeptides or to peptides shorter than the full length native or mature protein.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human).

Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Pharmaceutically acceptable” means physiologically tolerable, for either human or veterinary application.

As used herein, “pharmaceutical compositions” include formulations for human and veterinary use.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer.

Various solid phase peptide synthesis methods are known to those of skill in the art.

By “presensitization” is meant pre-administration of at least one innate immune system stimulator prior to challenge with an agent. This is sometimes referred to as induction of tolerance.

The term “prevent,” as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

A “prophylactic” treatment is a treatment administered to a subject who does not exhibit signs of a disease or exhibits only early signs of the disease for the purpose of decreasing the risk of developing pathology associated with the disease.

As used herein, “protecting group” with respect to a terminal amino group refers to a terminal amino group of a peptide, which terminal amino group is coupled with any of various amino-terminal protecting groups traditionally employed in peptide synthesis, Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, Cert-butyl, benzyl or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “receptor” is a compound that specifically binds to a ligand.

A “ligand” is a compound that specifically binds to a target receptor.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

As used herein, the term “reporter gene” means a gene, the expression of which can be detected using a known method. By way of example, the Escherichia coli lacZ gene may be used as a reporter gene in a medium because expression of the lacZ gene can be detected using known methods by adding the chromogenic substrate o-nitrophenyl-3-galactoside to the medium (Gerhardt et al., eds., 1994, Methods for General and Molecular Bacteriology, American Society for Microbiology, Washington, D.C., p. 574).

A “sample,” as used herein, refers preferably to a biological sample from a subject for which an assay or other use is needed, including, but not limited to, normal tissue samples, diseased tissue samples, sputum, mucus, phlegm, biopsies, cerebrospinal fluid, blood, serum, plasma, other blood components, gastric aspirates, throat swabs, pleural effusion, peritoneal fluid, follicular fluid, ascites, skin, hair, tissue, blood, plasma, cells, saliva, sweat, tears, semen, stools, Pap smears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. siRNA technology has been described (see, for example, U.S. Pat. Nos. 6,506,559, 7,056,704, 8,420,391 and 8,372,968, which are incorporated herein by reference in their entirety).

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds to”, as used herein, is meant when a compound or ligand functions in a binding reaction or assay conditions which is determinative of the presence of the compound in a sample of heterogeneous compounds.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal, Such animals include mammals, preferably a human.

As used herein, a “subject in need thereof” is a patient, animal, mammal, or human, who will benefit from the method of the presently disclosed subject matter.

As used herein, a “substantially homologous amino acid sequences” includes those amino acid sequences which have at least about 95% homology, preferably at least about 96% homology, more preferably at least about 97% homology, even more preferably at least about 98% homology, and most preferably at least about 99% or more homology to an amino acid sequence of a reference antibody chain. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C., Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it.

Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

The term to “treat,” as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

A “variant”, as described herein, refers to a peptide that differs from a reference peptide or to a segment of DNA that differs from the reference DNA. A “marker” or a “polymorphic marker”, as defined herein, is a variant. Alleles that differ from the reference are referred to as “variant” alleles.

A “vector” is a composition of matter which comprises an isolated nucleic acid and which can be used to deliver the isolated nucleic acid to the interior of a cell.

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasm ids (e.g., naked or contained in liposomes) and viruses that incorporate the recombinant polynucleotide.

The terms “microRNA” and “miRNA” are used interchangeably and refer to a nucleic acid molecule of about 17-24 nucleotides that is produced from a pri-miRNA, a pre-miRNA, or a functional equivalent. miRNAs are to be contrasted with short interfering RNAs (siRNAs), although in the context of exogenously supplied miRNAs and siRNAs, this distinction might be somewhat artificial. The distinction to keep in mind is that a miRNA is necessarily the product of nuclease activity on a hairpin molecule such as has been described herein, and an siRNA can be generated from a fully double-stranded RNA molecule or a hairpin molecule. Further information related to miRNAs generally, as well as a database of known published miRNAs and searching tools for mining the database can be found at the Wellcome Trust Sanger Institute miRBase: Sequences website, herein incorporated by reference. See also The microRNA Registry, Griffiths-Jones S., NAR, 2004, 32, Database Issue, D109-D111, herein incorporated by reference. miRNA technology has been described (see, for example, U.S. Pat. No. 7,960,359, 7,825,230, 7,825,229 and U.S. Pat. No. 7,592,441, which are incorporated herein by reference in their entirety).

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, and recombinantly produced RNA. Thus, RNAs include, but are not limited to mRNA transcripts, miRNAs and miRNA precursors, and siRNAs. As used herein, the term “RNA” is also intended to encompass altered RNA, or analog RNA, which are RNAs that differ from naturally occurring RNA by the addition, deletion, substitution, and/or alteration of one or more nucleotides, Such alterations can include addition of non-nucleotide material, such as to the end(s) of the RNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded, Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 300 basepairs, and in some embodiments between 15 and about 60 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin” or a “stem-loop structure.”

In describing the presently disclosed subject matter, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

As used herein, the term “substantially,” when referring to a value, an activity, or to an amount of a composition, mass, weight, temperature, time, volume, concentration, percentage, etc., is meant to encompass variations of in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions. For example, “substantially without depletion of mature B cells” can refer to a situation where basal levels of mature B cells in a subject, after administration of a composition of the presently disclosed subject matter, are at least 60%, or at least 75%, or at least 80%, or at least 85%, or at least 90%, or at least 95%, and, in certain cases, at least 99% of basal levels in the subject in the absence of administration of a composition of the presently disclosed subject matter.

### III. Embodiments

In some embodiments, the presently disclosed subject matter provides a composition for inhibiting a soluble B cell activating factor (BAFF) biological activity. In some embodiments, the composition comprises a reagent that interacts with a BAFF gene product, e.g. a BAFF polypeptide or a BAFF-encoding nucleic acid sequence; and a carrier, whereby inhibition of BAFF multimerization substantially without depletion of mature B cells is accomplished. In some embodiments, the composition comprises an anti-BAFF antibody that binds to a BAFF polypeptide and/or a nucleic acid that binds to a BAFF gene product. In some embodiments, the anti-BAFF antibody binds to an epitope present within a DE loop of a BAFF polypeptide and/or comprising a subsequence thereof, and depletion of mature B cells is substantially avoided when the antibody is administered. In some embodiments, the reagent is a peptide, such as a macrocyclic peptide as disclosed herein.

In some embodiments, the BAFF biological activity is selected from the group consisting of BAFF multimerization, binding of BAFF to a cognate receptor, inducing signal transduction mediated by a cognate receptor, and modulating growth of an abdominal aortic aneurysm (AAA). In some embodiments, the BAFF multimerization comprises multimerization of a trimer form of BAFF to a 60mer form of BAFF. In some embodiments, the cognate receptor is selected from the group consisting of BAFF receptor (BR3), CAML interactor (TACI), and B-cell maturation antigen (BCMA).

In some embodiments, provided is a pharmaceutical composition comprising the composition, optionally wherein the pharmaceutical composition is pharmaceutically acceptable for use in a human.

In some embodiments of the presently disclosed subject matter, an anti-BAFF antibody formulation is provided. In some embodiments, the anti-BAFF antibody formulation is prepared by immunizing a mammal with an antigen comprising an BAFF peptide or polypeptide. In some embodiments the BAFF antigen comprises the DE loop sequence, KVHVFGDELS (SEQ ID NO: 1), which is 10 amino acids long. In some embodiments, the sequence LVT, which is outside the C-terminus, is included to enhance solubility of the peptide. In some embodiments, the formulation inhibits BAFF multimerization substantially without depletion of mature B cells.

In some embodiments, the antigen comprises, consists essentially of, or consists of the amino acid sequence KVHVFGDELSLVT (SEQ ID NO: 2), and further optionally wherein the amino acid sequence KVHVFGDELSLVT (SEQ ID NO; 2) is conjugated to a carrier. In some embodiments, the mammal is selected from the group comprising a rabbit and a mouse. In some embodiments, the antigen comprises, consists essentially of, or consists of amino acid sequence KVHVFGDELSLVT (SEQ ID NO: 2) or the amino acid sequence KVHVFGDELS (SEQ ID NO; 1), optionally conjugated to keyhole limpet hemocyanin (KLH) via an N-terminal or a C-terminal cysteine addition. In some embodiments of the anti-BAFF antibody formulation, the mammal is a mouse and the anti-BAFF antibody is a monoclonal antibody. The DE peptide sequence is similar in mouse and human. In some embodiments, the antibodies are prepared against multiple DE peptides with variable amino acid lengths. By way of example, monoclonal Abs are generated against multiple lengths comprising DE loop and neighboring amino acids such as: LIQRKKVHVFGDELSLVTLF (SEQ ID NO: 3); IQRKKVHVFGDELSLVTL (SEQ ID NO: 4); KKVHVFGDELSL (SEQ ID NO: 5); LIQRKKVHVFGDELS (SEQ ID NO: 6); LIQRKKVHVFGDELSL (SEQ ID NO: 7); IQRKKVHVFGDELSLV (SEQ ID NO: 8); QRKKVHVFGDELSLVT (SEQ ID NO: 9); RKKVHVFGDELSLVTL (SEQ ID NO: 10); and LIQRKKVHVFGD (SEQ ID NO: 11). Bold indicates the DE loop amino acids.

In some embodiments, the presently disclosed subject matter provides a method for inhibiting a biological activity of a BAFF gene product. In some embodiments, the method comprises contacting the BAFF gene product with an effective amount of an inhibitor of BAFF. In some embodiments, the inhibitor comprises a reagent that interacts with a BAFF polypeptide or BAFF-encoding nucleic acid sequence, whereby inhibition of BAFF multimerization substantially without depletion of mature B cells is accomplished. In some embodiments, the composition comprises an anti-BAFF antibody that binds to a BAFF polypeptide and/or a nucleic acid that binds to a BAFF-encoding gene product. In some embodiments, the anti-BAFF antibody binds to an epitope present within a DE loop of a BAFF polypeptide and/or a subsequence thereof. In some embodiments, the nucleic acid comprises a siRNA, a miRNA or antisense oligonucleotide. In some embodiments, the nucleic acid comprises a siRNA, a miRNA or antisense oligonucleotide that binds a BAFF-encoding nucleotide sequence as disclosed herein, whether DNA or RNA, such as via interaction of complementary sequences to form double stranded structures. Representative BAFF-encoding sequences are disclosed in Table 2. the formulation inhibits BAFF multimerization substantially without depletion of mature B cells. In some embodiments, the inhibitor comprises a peptide, such as a macrocyclic peptide as disclosed herein.

In some embodiments, the BAFF biological activity is selected from the group comprises BAFF multimerization, binding of BAFF to a cognate receptor, inducing signal transduction mediated by a cognate receptor, and modulating growth of an abdominal aortic aneurysm (AAA). In some embodiments, BAFF multimerization comprises multimerization of a trimer form of BAFF to a 60mer form of BAFF. In some embodiments, the cognate receptor is selected from the group comprises BAFF receptor (BR3), CAML interactor (TACI), and B-cell maturation antigen (BCMA). In some embodiments, the anti-BAFF antibody binds to an epitope present within a DE loop of a BAFF polypeptide and/or comprising a subsequence thereof, to thereby inhibit BAFF multimerization, inhibit binding of BAFF to a cognate receptor, and/or inhibit signal transduction mediated by a cognate receptor. In some embodiments, the biological activity of the BAFF gene product is associated with growth of an abdominal aortic aneurysm (AAA).

Also provided in accordance with the presently disclosed subject matter are methods for inhibiting growth of an abdominal aortic aneurysm (AAA) in a subject. In some embodiments, the method comprises administering to the subject an effective amount of a composition that inhibits the activity of BAFF. In some embodiments, the inhibitor comprises a reagent that interacts with a BAFF polypeptide or BAFF-encoding nucleic acid sequence, whereby inhibition of BAFF multimerization substantially without depletion of mature B cells is accomplished. In some embodiments, the composition comprises an anti-BAFF antibody that binds to a BAFF polypeptide and/or a nucleic acid that binds to a BAFF-encoding nucleic acid sequence. In some embodiments, the anti-BAFF antibody binds to an epitope present within a DE loop of a BAFF polypeptide and/or a subsequence thereof. In some embodiments, the nucleic acid comprises a siRNA, a miRNA or antisense oligonucleotide. In some embodiments, the nucleic acid comprises a siRNA, a miRNA or antisense oligonucleotide that binds a BAFF-encoding nucleotide sequence as disclosed herein, whether DNA or RNA, such as via interaction of complementary sequences to form double stranded structures. Representative BAFF-encoding sequences are disclosed in Table 2.

Provided in accordance with the presently disclosed subject matter are novel reagents that can be used for treatment of AAA and other B cell related diseases. In some embodiments, the reagent interacts with a BAFF polypeptide or BAFF-encoding nucleic acid sequence, whereby inhibition of BAFF multimerization substantially without depletion of mature B cells is accomplished. Therapeutically effective amounts of such reagents are administered to a subject in need of treatment, such as treatment for AAA and other B cell related diseases. BAFF 60mer is more active than BAFF 3mer. The 3mer binds only to BAFF receptor BR3, whereas, the 60mer binds to BR3, TACI and BCMA. The present data demonstrates that blocking BR3 via anti-BR3 IgG1 aggravates aortic aneurysm growth in mice. This suggests that 60mer binding to TACI and BCMA promotes aneurysm growth. Therefore, regents in accordance with the presently disclosed subject matter inhibit BAFF 60mer formation. There reagents include an antibody targeting BAFF DE loop (which is required for 60mer formation) and macrocyclic peptides, which will bind to the DE loop as a competitive inhibitor for 60mer formation. A representative cyclic peptide is DE1akm, IQRKKVHVFGDELSLVTL (SEQ ID NO: 12), head to tail cyclization. The DE1 akm peptide is designed to retain a part of and ‘E’ beta-sheets and the DE loop. This structure should serve as a competitive inhibitor. Another representative cyclic peptide is DE3akm, VHVFGDEL (SEQ ID NO: 13), head to tail cyclization. Another representative peptide is DE4akm, Ac-RKKVHVFGDELSLV-NH2 (SEQ ID NO: 14). These peptides were prepared by a synthetic route, but can be prepared by a suitable approach as disclosed herein and as would be apparent to one of ordinary skill in the art up on a review of the instant disclosure as can suitable modified versions, fragments, and/or variants of these peptides. The presently disclosed reagents will not only help in the treatment of AAAs, but also in the treatment of B cell related diseases, such as lupus, cardiovascular diseases, type 1 and type 2 diabetes, and B cell lymphomas.

The presently disclosed subject matter provides compositions and methods for, inter aha, inhibiting BAFF multimerization, inhibiting BAFF binding to cognate receptors, inhibiting BAFF activity, and inhibiting BR3 signal transduction, and preventing AAA growth. In some embodiments, BAFF is BAFF 60 mer. In some embodiments, the inhibitor comprises a reagent that interacts with a BAFF polypeptide or BAFF gene product whereby inhibition of BAFF multimerization substantially without depletion of mature B cells is accomplished. In some embodiments, the inhibitor is an antibody. In some embodiments, an antibody is used to inhibit BAFF multimerization. In some embodiments, an antibody of the presently disclosed subject matter includes, but is not limited to, monoclonal antibodies, single chain antibodies, synthetic antibodies, humanized antibodies, and chimeric antibodies, and biologically active fragments and homologs thereof. In some embodiments, the antibody is a monoclonal antibody. In some embodiments, the antibody is a polyclonal antibody. In some embodiments, the antibody is a monoclonal antibody. In some embodiments, the antibody is purified. In some embodiments, the antibody is directed against BAFF. In some embodiments, the antibody, or a fragment thereof, is directed against the DE loop. In some embodiments, an antibody or peptide of the presently disclosed subject matter can be administered to a subject at a dosage from about 0.01 mg/kg to about 100 mg/kg, about 0.1 mg/kg to about 75 mg/kg, about 0.5 mg/kg to about 50 mg/kg, about 1.0 mg/kg to about 25 mg/kg, about 2.0 mg/kg to about 20 mg/kg, about 3.0 mg/kg to about 15 mg/kg, about 4.0 mg/kg to about 10 mg/kg, or about 5.0 mg/kg to about 7.5 mg/kg. The presently disclosed subject matter further encompasses the administration of unit doses, which can be, for example, 1, 5, 10, 20, 25, 30, 35, 40, 45, 50, 55, 60, 65, 70, 75, 80, 85, 90, 95, 100, 125, 150, 200, 500, 1,000, or 5,000 mg.

In some embodiments, an antibody directed against BAFF is useful for inhibiting AAA growth. In one aspect, the antibody is a monoclonal antibody.

In some embodiments, an anti-BAFF antibody of the presently disclosed subject matter inhibits BAFF binding to BAFF-R and TACI. In some embodiments, the antibody blocks BAFF activity.

In some embodiments, the presently disclosed subject matter provides compositions and methods for regulating BAFF-BR3 signaling. In some embodiments, it is useful for treating AAA. In some embodiments, it is useful for inhibiting AAA growth. Since there is a role for B cell and BAFF implicated in cardiovascular diseases, and in type 1 and type 2 diabetes, in some embodiments, the presently disclosed subject matter provides for treatment of these diseases and other B cell related diseases.

The presently disclosed subject matter adds to the understanding of regulation of B cell function by BAFF multimerization and role of B cells in AAA. Novel reagents and methods are provided, which provide for development of treatment strategies for AAA and other B cell-related diseases such as lupus, cardiovascular diseases, and type 1 and type 2 diabetes.

In some embodiments, the BAFF biological activity is selected from the group comprises BAFF multimerization, binding of BAFF to a cognate receptor, inducing signal transduction mediated by a cognate receptor, and modulating growth of an abdominal aortic aneurysm (AAA). In some embodiments, BAFF multimerization comprises multimerization of a trimer form of BAFF to a 60mer form of BAFF. In some embodiments, the cognate receptor is selected from the group comprises BAFF receptor (BR3), CAVIL interactor (TACI), and B-cell maturation antigen (BCMA). In some embodiments, the anti-BAFF antibody binds to an epitope present within a DE loop of a BAFF polypeptide and/or comprising a subsequence thereof, to thereby inhibit BAFF multimerization, inhibit binding of BAFF to a cognate receptor, and/or inhibit signal transduction mediated by a cognate receptor. In some embodiments, the biological activity of the BAFF gene product is associated with growth of an abdominal aortic aneurysm (AAA).

Any suitable route of administration of an effective amount of a BAFF inhibitor as would be apparent to one of ordinary skill in the art up on a review of the presently disclosure can be taken. Representative approaches include but are not limited to intraperitoneal, intravenous, intramuscular, administration via application to an AAA (for example, Pluronic gel on the top of the AAA). In cardiovascular and diabetes treatments, an anti-BAFF therapeutic be injected intraperitoneally or intravenously, for example.

In some embodiments, BAFF inhibitors or anti-BAFF therapeutics include aptamers, macrocyclic peptides, and small molecule inhibitors. A representative cyclic peptide is DE1akm, IQRKKVHVFGDELSLVTL (SEQ ID NO: 12), head to tail cyclization. The DE1akm peptide is designed to retain a part of ‘D’ and ‘E’ beta-sheets and the DE loop. This structure should serve as a competitive inhibitor. Another representative cyclic peptide is DE3akm, VHVFGDEL (SEQ ID NO: 13), head to tail cyclization. Another representative peptide is DE4akm, Ac-RKKVHVFGDELSLV-NH2 (SEQ ID NO: 14). These peptides were prepared by a synthetic route but can be prepared by a suitable approach as disclosed herein and as would be apparent to one of ordinary skill in the art up on a review of the instant disclosure as can suitable modified versions, substantially homologous amino acid sequences, fragments, and/or variants of these peptides. In some embodiments, such reagents bind to the DE loop as a competitive inhibitor for 60mer formation. Techniques for designing such reagents (e.g., macrocyclic peptides) are described elsewhere herein, including in Example 10.

TABLE 2
GENBANK ® Accession Nos.

| BAFF | | BR3 | |
|---|---|---|---|
| Also called tumor necrosis factor | | Also called tumor necrosis factor | |
| ligand superfamily member 13B | | receptor superfamily member 13C | |
| (TNFSF13B) | | (TNFRSF13C) | |
| Nucleic Acid | Amino Acid | Nucleic Acid | Amino Acid |

Homo sapiens

| | | | |
|---|---|---|---|
| NM_006573.4 | NP_006564.1 | NM_052945.3 | NP_443177.1 |

Mus musculus

| | | | |
|---|---|---|---|
| NM_033622.2 | NP_296371.1 | NM_033622.2 | NP_296371.1 |

Pan troglodytes

| | | | |
|---|---|---|---|
| NM_001328319.1 | NP_001315248.1 | XM_016939298.1 | XP_016794787.1 |

Gorilla gorilla gorilla

| | | | |
|---|---|---|---|
| XM_019039540.1 | XP_018895085.1 | XM_019018034.1 | XP_018873579.1 |

Macaca mulatto

| | | | |
|---|---|---|---|
| XM_001082247.3 | XP_001082247.1 | XM_001101623.3 | XP_001101623.2 |

Canis lupus familiaris

| | | | |
|---|---|---|---|
| NM_001161710.2 | NP_001155182.1 | XM_843968.5 | XP_849061.2 |

Felis catus

| | | | |
|---|---|---|---|
| XM_023253118.1 | XP_023108886.1 | XM_023257540.1 | XP_023113308.1 |

Bos taurus

| | | | |
|---|---|---|---|
| NM_001114506.1 | NP_001107978.1 | NM_001114506.1 | NP_001107978.1 |

Sus scrofa

| | | | |
|---|---|---|---|
| XM_005668530.3 | XP_005668587.1 | XM_021091439.1 | XP_020947098.1 |

Equus caballus

| NM_001242445.1 | NP_001229374.1 | XM_023631009.1 | XP_023486777.1 |
|---|---|---|---|
| TACI | | BCMA | |
| Also called tumor necrosis factor | | Also called tumor necrosis factor | |
| receptor superfamily member 13B | | receptor superfamily member 17 | |
| (TNFRSF13B) | | (TNFRSF17) | |
| Nucleic Acid | Amino Acid | Nucleic Acid | Amino Acid |

Homo sapiens

| | | | |
|---|---|---|---|
| NM_012452.2 | NP_036584.1 | NM_001192.3 | NP_001183.2 |

Mus musculus

| | | | |
|---|---|---|---|
| NM_021349.2 | NP_067324.1 | XM_006521989.3 | XP_006522052.1 |

Pan troglodytes

| | | | |
|---|---|---|---|
| XP_001161361.3 | XP_001161361.3 | XM_523298.5 | XP_523298.2 |

Gorilla gorilla gorilla

| | | | |
|---|---|---|---|
| XM_004042197.1 | XP_004042245.1 | XM_004057229.2 | XP_004057277.1 |

Macaca mulatta

| | | | |
|---|---|---|---|
| XM_015118722.1 | XP_014974208.1 | XM_001106892.3 | XP_001106892.1 |

Canis lupus familiaris

| | | | |
|---|---|---|---|
| XM_005620177.3 | XP_005620234.1 | XM_005621530.3 | XP_005621587.1 |

Felis catus

| | | | |
|---|---|---|---|
| XM_023244016.1 | XP_023099784.1 | XM_006942320.2 | XP_006942382.1 |

Bos taurus

| | | | |
|---|---|---|---|
| XM_024980816.1 | XP_024836584.1 | XM_002697966.5 | XP_002698012.2 |

Sus scrofa

| | | | |
|---|---|---|---|
| XM_021068044.1 | XP_020923703.1 | XM_003124587.6 | XP_003124635.1 |

Equus caballus

| | | | |
|---|---|---|---|
| XM_005598001.2 | XP_005598058.2 | XM_014730110.2 | XP_014585596.1 |

Antibodies refer to polypeptides substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

A variety of methods for producing polyclonal and monoclonal antibodies are known in the art. See, e.g., Goding, MONOCLONAL ANTIBODIES; PRINCIPLES AND PRACTICE, Academic Press, 2nd Edition (1986); and Harlow & Lane. A monoclonal antibody directed against or reactive with, for example, human cells expressing a desired antigen is obtained by using combinations of immunogens to immunize mice and screening hybridoma supernatant against cells which express the desired antigen or by a screening assay designed to be specific for monoclonal antibodies directed against the antigen of interest. Useful cell lines for screening for the antibodies of the presently disclosed subject matter are readily available or obtained. Such cells include the Burkitt's lymphoma cell lines Daudi, and Raji.

Recombinant DNA methodologies can be used to synthesize antibodies of the presently disclosed subject matter. For example, an antibody portion of an immunotoxin for use in humans is a “humanized” antibody against a cell antigen which contains murine complementarity-determining regions (CDRs) combined with human variable region frameworks and human constant regions.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source.

The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos. 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369). Detailed methods for preparation of chimeric (humanized) antibodies can be found in U.S. Pat. No. 5,482,856.

In another embodiment, the presently disclosed subject matter provides for fully human antibodies. Human antibodies consist entirely of characteristically human polypeptide sequences. The human antibodies of the presently disclosed subject matter can be produced in using a wide variety of methods (see, e.g., Larrick et al, U.S. Pat. No. 5,001,065, for review).

The antibody moieties of the presently disclosed subject matter can be single chain antibodies. In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibodies directed against proteins, polypeptides, or peptide fragments thereof of the presently disclosed subject matter may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the presently disclosed subject matter.

Antibody fragments which contain the idiotype of the antibody molecule can be generated by known techniques. For example, such fragments include but are not limited to: the F(ab′)2 fragment which can be produced by pepsin digestion of the antibody molecule; the Fab′ fragments which can be generated by reducing the disulfide bridges of the F(ab′)2 fragment; the Fab fragments which can be generated by treating the antibody molecule with pepsin and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al, (1992, Critical Rev. in Immunol. 12(3,4):125-168) and the references cited therein. Further, the antibody of the presently disclosed subject matter may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from miRNA which is isolated from cells, e.g., the hybridoma, which express the desired protein to be expressed on the phage surface, e.g., the desired antibody, cDNA copies of the miRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example. in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell, Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57; 191-280), Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the presently disclosed subject matter should not be construed to be limited solely to the generation of phage encoding Fab antibodies.

Rather, phage which encode single chain antibodies (scFv/phage antibody libraries) are also included in the presently disclosed subject matter. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The presently disclosed subject matter should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present presently disclosed subject matter may include, but are not limited to, polyclonal, monoclonal, chimeric (i.e., “humanized”), and single chain (recombinant) antibodies, Fab fragments, and fragments produced by a Fab expression library.

The peptides of the presently disclosed subject matter may be readily prepared by standard, well-20established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyimide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions that will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method that utilized Cert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

To ensure that the proteins or peptides obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide can be purified to remove contaminants. In this regard, it will be appreciated that the peptide will be purified to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica, A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure peptide obtained as described herein may be purified by following known procedures for protein purification, wherein an immunological, enzymatic or other assay is used to monitor purification at each stage in the procedure.

Protein purification methods are well known in the art, and are described, for example in Deutscher et al. (ed., 1990, Guide to Protein Purification, Harcourt Brace Jovanovich, San Diego),

Peptide Modification and Preparation

Peptide preparation is described elsewhere herein, including in the Examples. A representative cyclic peptide is DE1akm, IQRKKVHVFGDELSLVTL (SEQ ID NO: 12), head to tail cyclization. The DE1akm peptide is designed to retain a part of ‘D’ and ‘E’ beta-sheets and the DE loop. This structure should serve as a competitive inhibitor. Another representative cyclic peptide is DE3akm, VHVFGDEL (SEQ ID NO: 13), head to tail cyclization. Another representative peptide is DE4akm, Ac-RKKVHVFGDELSLV-NH2 (SEQ ID NO: 14). These peptides were prepared by a synthetic route, but can be prepared by a suitable approach as disclosed herein and as would be apparent to one of ordinary skill in the art up on a review of the instant disclosure as can suitable modified versions, substantially homologous amino acid sequences fragments, and/or variants of these peptides.

It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to 10 encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide.

For example, suitable N-terminal blocking groups can be introduced by alkylation or acylation of the N-terminus. Examples of suitable N-terminal blocking groups include C1-05 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

Modifications (which do not normally alter primary sequence) include in vivo, or in vitro chemical derivatization of polypeptides, e.g., acetylation, or carboxylation.

Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring or nonstandard synthetic amino acids. The peptides of the presently disclosed subject matter are not limited to products of any of the specific exemplary processes listed herein. The presently disclosed subject matter includes the use of beta-alanine (also referred to as β-alanine, β-Ala, bA, and βA.

Peptides useful in the presently disclosed subject matter, such as standards, or modifications for analysis, may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyimide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters.

Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well-known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-meth benzhydrylamine (MBNA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HD treatment releases a peptide bearing an N-methy amidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide may be purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high performance liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

Amino Acid Substitutions

In certain embodiments, the disclosed methods and compositions may involve preparing peptides with one or more substituted amino acid residues. In various embodiments, the structural, physical and/or therapeutic characteristics of peptide sequences may be optimized by replacing one or more amino acid residues. Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid residues or may comprise amino acids which are all in the D-form. Retro-inverse forms of peptides in accordance with the presently disclosed subject matter are also contemplated, for example; inverted peptides in which all amino acids are substituted with D-amino acid forms. As used herein, the term “variant” compasses such substitutions.

The skilled artisan will be aware that, in general; amino acid substitutions in a peptide typically involve the replacement of an amino acid with another amino acid of relatively similar properties (i.e.; conservative amino acid substitutions). The properties of the various amino acids and effect of amino acid substitution on protein structure and function have been the subject of extensive study and knowledge in the art. As used herein, the term “variant” compasses such substitutions.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine. norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo; or iodo) or alkoxy-substituted forms of the previous listed aromatic amino acids, illustrative examples of which are: 2-,3-or 4-am inophenylalanine, 2-,3- or 4-chlorophenylalanine; 2-,3- or 4-methylphenylalanine, 2-,3-or 4-methoxyphenylalanine, 5-amino-, 5-chloro-, 5-methyl-or 5-methoxytryptophan, 2′-, 3′-; or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N, N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); ieucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5), in making conservative substitutions, the use of amino acids whose hydropathic indices are within +/− 2 is preferred, within +1-1 are more preferred, and within +/−0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3,0); serine (+0.3); asparagine (+0.2); glutamine (+0.2); glycine (0); threonine (−0.4); proline (−0.5,+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see, e,g., Chou & Fasman, 1974, Biochemistry, 13:222-245; 1978, Ann. Rev. Biochem., 47: 251-276; 1979, Biophys. J., 26:367-384).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) leu, ile, val; Arg (R) gin, asn, lys; Asn (N) his, asp, lys, arg. gln; Asp (D) asn, glu; Cys (C) ala, ser; Gln (Q) glu, asn; Glu (E) gin, asp; Gly (G) ala; His (H) asn, gin, lys, arg; Ile (I) val, met, ala, phe, leu; Leu (L) val, met, ala, phe, ile; Lys (K) gin, asn, arg; Met (M) phe, ile, leu; Phe (F) leu, vel, ile, ala, tyr; Pro (P) ala; Ser (S), thr; Thr (T) ser; Trp (W) phe, tyr; Tyr (Y) trp, phe, thr, ser; Val (V) ile, leu, met; phe, ala.

Other considerations for amino acid substitutions include whether or not the residue is located in the interior of a protein or is solvent exposed. For interior residues, conservative substitutions would include; Asp and Asn; Ser and Thr; Ser and Ala; Thr and Ala; Ala and Gly; He and Val; Val and Leu; Leu and He; Leu and Met; Phe and Tyr; Tyr and Trp. (See, e.g., PROWL Rockefeller University website). For solvent exposed residues, conservative substitutions would include: Asp and Asn; Asp and Glu; Glu and Gin; Glu and Ala; Gly and Asn; Ala and Pro; Ala and Gly; Ala and Ser; Ala, and Lys; Ser and Thr; Lys and Arg; Val and Leu; Leu and He; He and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Disler matrix (Idem.)

In determining amino acid substitutions, one may also consider the existence of intermolecular or intramolecular bonds, such as formation of ionic bonds (salt bridges) between positively charged residues (e.g., His. Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Pharmaceutical Compositions and Administration

The presently disclosed subject matter is also directed to methods of administering the compounds, including but not limited to antibodies and nucleotide sequences, of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method of treating a subject in need of such treatment is provided. The method comprises administering a pharmaceutical composition comprising at least one compound of the presently disclosed subject matter to a subject in need thereof. Compounds identified by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is provided include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Genaro, ed., 1985, Remington's Pharmaceutical Sciences, Mack Publishing Co., Easton, P A, which is incorporated herein by reference.

Typically, dosages of the compound of the presently disclosed subject matter which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In one aspect, the dosage of the compound will vary from about 1 mg to about 10 g per kilogram of body weight of the animal. In another aspect, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated; the type and age of the animal, etc.

Suitable preparations include injectables, either as liquid solutions or suspensions, however; solid forms suitable for solution in, suspension in, liquid prior to injection, may also be prepared. The preparation may also be emulsified, or the polypeptides encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants.

The presently disclosed subject matter also includes a kit comprising a composition of the presently disclosed subject matter and an instructional material which describes adventitially administering the composition to a cell or a tissue of a subject. In another embodiment, this kit comprises a (preferably sterile) solvent suitable for dissolving or suspending the composition of the presently disclosed subject matter prior to administering the compound to the subject.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains the multimeric peptide of the presently disclosed subject matter or be shipped together with a container which contains the peptide. Alternatively, the instructional material may be shipped separately from the container with the intention that the instructional material and the compound be used cooperatively by the recipient.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

With reference to the following Examples and without further description, it is believed that one of ordinary skill in the art can, using the preceding description and the following illustrative examples, make, utilize, and/or practice the presently disclosed and claimed subject matter. Therefore, the Examples should be construed to encompass any and all variations which become evident as a result of the teaching provided herein.

### Example 1

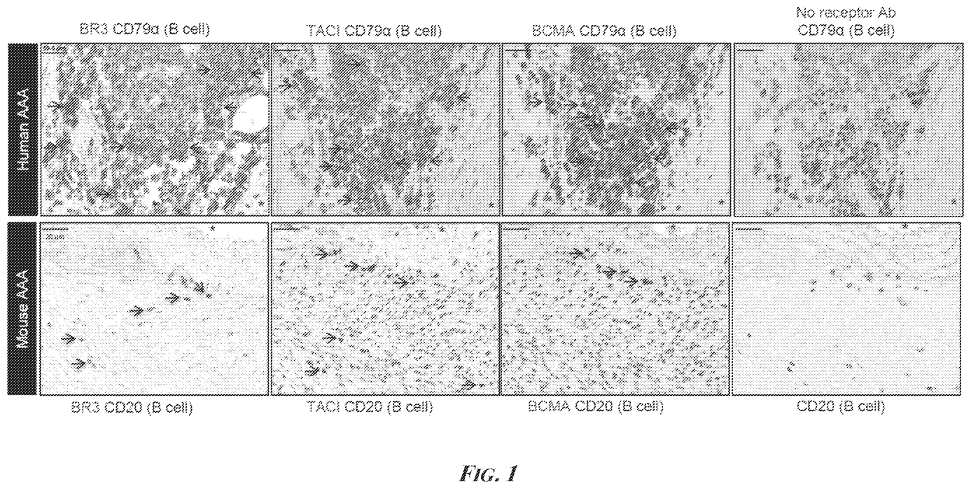

Naïve B cells are metabolically quiescent, whereas, antibody producing plasma cells are metabolically active. BAFF regulates differentiation of naive B cells to plasma cells. Immunohistochemistry was performed on human AAA (huAAA) and mouse elastase perfusion-induced AAA (moAAA) sections and images were acquired on a light microscope. See FIG. 1. The presence of BR3 expressing B cells in the milieu of BAFF in human AAA tissues was demonstrated. Since the 60mer is more active than 3mer in B cell survival assays and BAFF (3mer or 60mer unknown) can promote B cell and without a desire to be bound by any particular theory of operation, it is hypothesized that the 60mer, but not 3mer treatment induces differentiation of naïve B cells to antibody producing plasma cells via inducing both NF-kB1 and NE-kB2 signaling and increasing energy metabolism.

### Introduction to Examples 2-9

Examples 2-9 relate to the elucidation of a role for BAFF in neutrophil-B cell crosstalk and AAA formation; the determination of the functional heterogeneity of infiltrated B cells and neutrophils in murine AAA tissues by single cell RNA sequencing; the evaluation of hypotheses that BAFF 60mer, but not the 3mer, activates B cells by binding to BR3 and TACI receptors and promotes AAA formation; the examination of whether BAFF produced from neutrophils is required for B cell activation and AAA formation; and the determination of the role of IgG1 and IgG2 in FcγRIII-mediated aortic inflammation and AAA formation.

Examples 2-9 also relate to the examination of inhibition of BAFF 60mer formation in the attenuation of neutrophil-B cell inflammatory crosstalk, B2 cell activation and AAA formation. Based on encouraging results from the use of the presently disclosed polyclonal anti-DE3 Ab, monoclonal anti-DE Abs are provided, which are highly specific and reproducible in activity. The monoclonal Abs are validated using biochemical and biophysical methods. The anti-DE Abs' activities to suppress the growth of established murine AAAs is also examined. The effectiveness of anti-DE Abs with available anti-BAFF biologics in attenuating B cell activation in human neutrophil-B cell co-cultures is also provided.

Examples 2-9 also relate to identifying the BAFF 60mer, not the BAFF molecule, as a therapeutic target in vascular diseases. Furthermore, in accordance with some aspects, the presently disclosed anti-DE Ab does not deplete B cells and hence is advantageous over Belimumab, a known anti-BAFF Ab used for treatment of systemic lupus erythematosus.

### Example 2

### Neutrophils can Communicate with B Cells Via BAFF

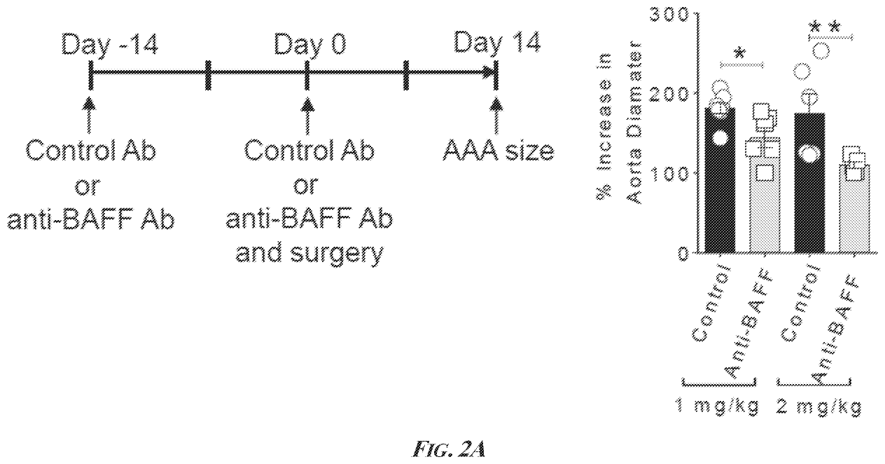

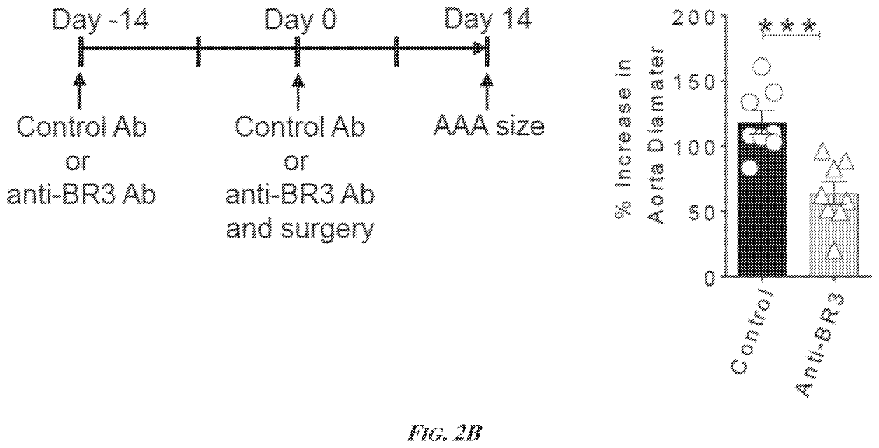

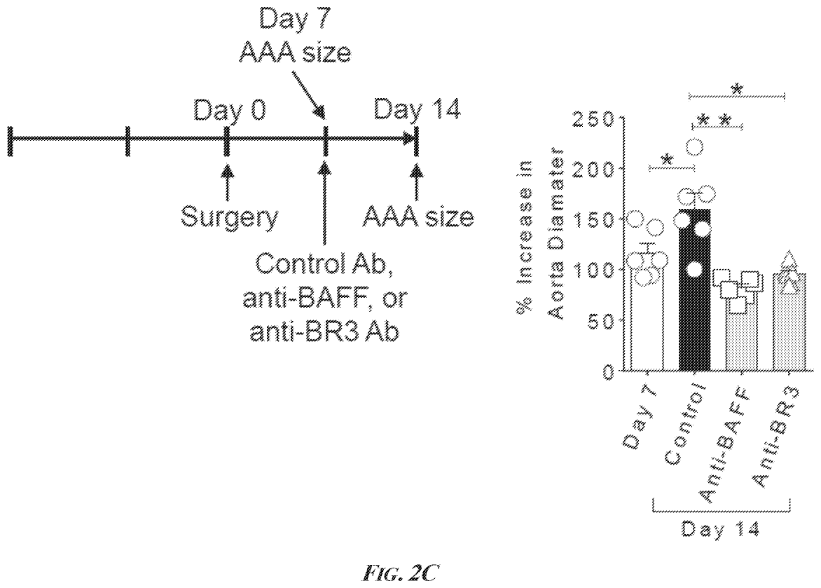

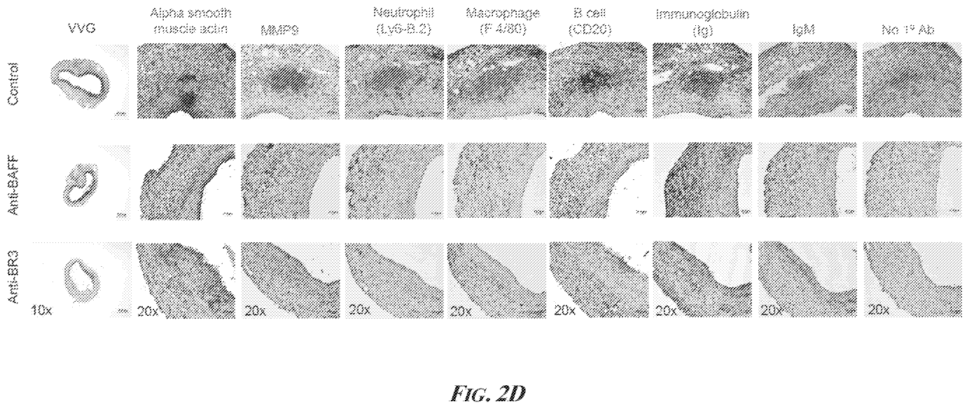

Murine models of AAA are known to recapitulate the early stages of human AAA. B cells were identified and B cell subtypes were quantified in elastase perfusion model of murine AAAs⁵. In a treatment strategy, AAA was induced in three groups of WT mice via topical elastase model and 7 days after the AAA induction, mice were treated intravenously with a control Ab, anti-BAFF, or anti-BR3 Ab (n=6/group). Seven days after the Ab treatment, AAA size was determined. As a control, AAA size was determined in 7 days after AAA induction (group Day 7, n=6). Serial aortic cross-sections from the treatment group were stained to examine degradation of elastin layer (VVG), smooth muscle cell layer (alpha smooth muscle actin), expression of MMP9, presence of neutrophils, macrophages, B cells, immunoglobulin and IgM. As a control, an aortic section was stained without the primary(1°) Ab. Hematoxylin (blue/purple) staining was used to identify the nuclei. Values were expressed as means+SEM.*, p<0.05; **,p<0.01: and ***, p<0.001 by Student's t-test (FIGS. 2A-2D). In AAA tissues, B2 cell count s 20 fold higher than B1 cells. ˜4000 B2 cells out of ˜100,000 infiltrated immune cells in murine AAAs.

### Example 3

### BAFF 3mer and 60mer can Differentially Regulate B Cell Function

After synthesis, BAFF is expressed as a membrane-bound protein and cleaved to a soluble form¹⁸, which is required for survival of naive B cells, B2 cells, and antibody producing plasma cells. Interestingly, soluble BAFF (sal 34-285) exists as a 3mer at pH 5_6 buffer and as 60mer at pH≥7.4 buffer¹¹. BAFF is secreted as a 3mer and BAFF 60mer formation requires the solvent accessible DE loop. Mutation in the Histidine 218 to Alanine (H218A) of DE loop inhibits multimerization of the 3mer to the 60mer. Moreover, recombinant myc-tagged BAFF and Flag-tagged BAFF exist as 3mers, even at pH≥7.4 and used as BAFF 3mer in cell culture experiments¹⁹.

The binding affinity of 3mer to the BR3 receptor is similar to that of 60mer, however, the 60mer is more active than 3mer in inducing proliferation of activated B cells¹⁹. This is because while the BAFF 3mer can only bind to BR3 receptor, the 60mer can bind to BR3, TACI and BCMA receptors on B cells. Expression of BR3 and TACI receptors are required for differentiation of B2 cells to antibody producing plasma B cells in response to BAFF²⁰, whereas, BCMA is required for survival of plasma B cells. Mice genetically deficient in BAFF or BR3 exhibit a similar immune phenotype, such as, (1) lower numbers of mature B cells, (2) a lower number of B2 cells in spleen, (3) lower expression levels of CD21 and CD23 in B cells (B2 cell markers), and (4) reduced antigen-specific antibody response^{21, 22}. To study the role of BAFF in wild-type mice, multiple BAFF neutralizing anti-BAFF Abs are available. One of the Abs protects mice from diet-induced insulin resistance; however, the level of depletion of various B cell sub-types is not clear²³. Sandy-2, another anti-BAFF Ab, depletes splenic B cells from 57% to 22%²⁴.

In mouse plasma (pH is 7.4), only 4% of the total BAFF is 60mer, suggesting high concentration of BAFF 3mer is required for 60mer formation. Injection of 40 μg/mouse (i.p. daily) of H218A BAFF (3mer) to BAFF^{-/-} mice, restores peripheral B cell populations and antibody responses¹⁶. However, injection of the 60mer not only increases the level of CD23 expression on B cells, but also increases the number of B2 cells. So far, little is known about BAFF-induced genes. Furthermore, prior to the present disclosure, the roles of the 3mer and the 60mer in vivo were unknown.

### Example 4

### Differentiation and Survival of B Cells are Dependent on NF-KB Signaling and Intracellular Metabolic Reprogramming

The role of NF-kB in B cell survival and differentiation is known. BAFF binding to BR3 on B cell surface degrades TRAF3 leading to NIK accumulation and phosphorylation of IKKα and p100 (NF-kB2 signaling). Furthermore, BAFF can activate both the NF-kB1 and -kB2 pathways via the anti-apoptotic protein Bcl10²⁵. However, TACI activation can suppress BR3-mediated NF-kB2 signaling²⁶. However, in these studies, it is not clear if the BAFF used was the 3mer or the 60mer, and, what is the outcome of BAFF binding to B2 cells (express both BR3 and TACO and to plasma cells (express BCMA).

Aerobic glycolysis (pyruvate from glycolysis is directed towards lactate production) and oxidative phosphorylation (OXPHOS, pyruvate is used in mitochondrial citric acid cycle) are the two major pathways of energy production in the cell. Choice of one of the pathways or both significantly affects B cell function e.g. antibody production by plasma cells require aerobic glycolysis²⁷. Recently, Lam et al. demonstrated long-lived plasma cells undergo significantly higher OXPHOS compared to short-lived plasma cells and utilize mitochondrial pyruvate import for long-term survival and antibody production²⁸. However, prior to the instant disclosure, it was unknown if the 60mer induces a unique metabolic signature in B2 cells to activate and differentiate in to plasma cells.

### Example 5

### IgGs can Induce Vascular Inflammation

Supplementing the B cell deficient muMT mice with a mouse polyclonal IgG exacerbates aneurysm formation²⁹. Moreover, IgG stimulates IL-6 and MMP9 secretion in human AAA tissue cultures. These results suggest that IgGs nonspecifically bind to aortic wall and exacerbate inflammation. However, IgGs are normally present in the circulation of human and mice, why do not they induce AAA? The Fc region of IgGs can bind to Fcγ receptors (FcγRs) and activation of FcγRs contributes to the vascular pathogenesis¹⁰. In inflamed vascular wall, FcγRs are expressed by smooth muscle cells (SMCs), endothelial cells and infiltrated monocytes/macrophages, dendritic cells, neutrophils, mast cells and B cells. Particularly, aberrant expression of FcγRIII (also known as CD16) promotes atherosclerosis³⁰⁻³², whereas, activation of FcγRs promotes MMP secretion by macrophages³³. Our BAFF neutralization study demonstrates significant depletion of plasma IgG1 and IgG2 and healthy aortic morphology even with the presence of FcγRIII in aortic wall. While it is not desired to be bound by any particular theory of operation, it is hypothesized that IgG1 and IgG2 are necessary for FcγRIII-mediated inflammation, aortic wall degradation, and AAA formation.

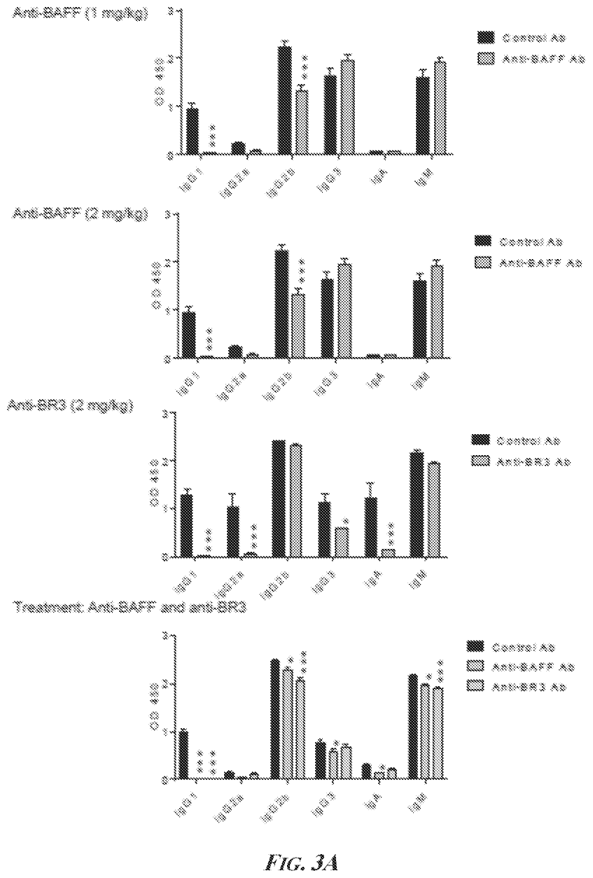

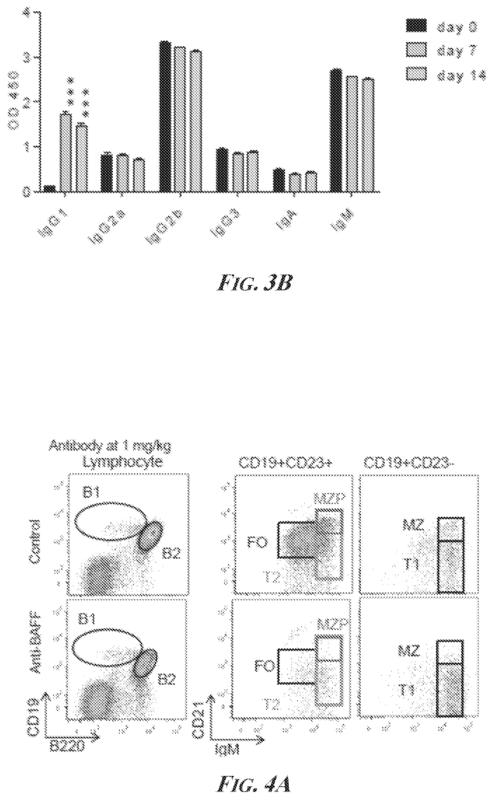

Data described herein suggest that both neutrophils and B cells are required for aortic inflammation and AAA formation. Neutrophils can activate and differentiate B cells to antibody producing plasma cells via secreting BAFF. The present results demonstrate that BAFF depletion attenuates AAA formation (FIGS. 2A-2C) despite infiltration of neutrophils and B cells in AAA. Furthermore, BAFF depletion significantly decreases the plasma level of only two of the immunoglobulins IgG1 and IgG2 [FIGS. 3A and 3B] and prevents IgG deposition in aorta (FIG. 2D). These results suggest a possible role of B cell-helper neutrophils producing BAFF to activate B cells, which further secrete pathogenic IgGs (IgG1 and IgG2) in AAA tissues.

The present results demonstrate that B cells in AAA express BR3 & TACI (FIG. 1), neutrophils localize close to B cells and plasma cells are abundant in AAA tissues (FIG. 11). Furthermore, the 60mer, but not the 3mer, activates B cells. Therefore, it is tested whether neutrophil-secreted BAFF forms 60mer, which activates B2 cells, produce pathogenic IgGs that damage the aorta and promote AAA formation.

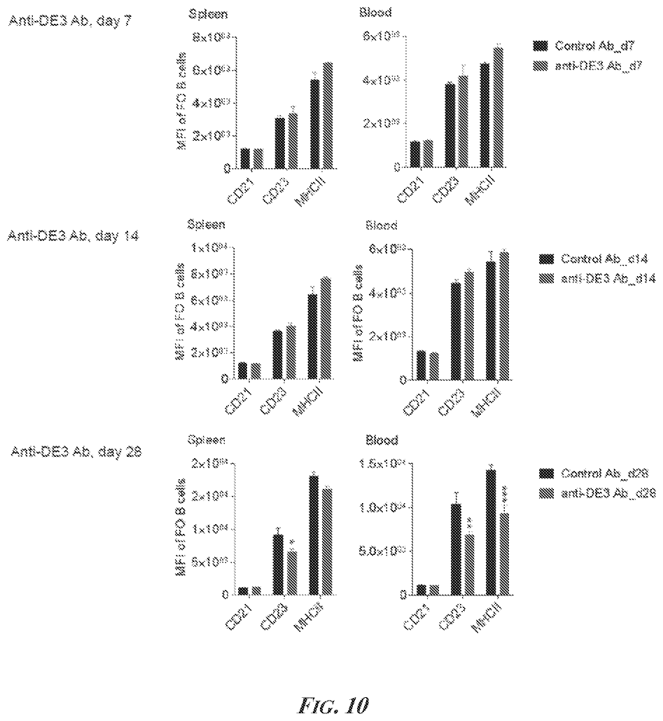

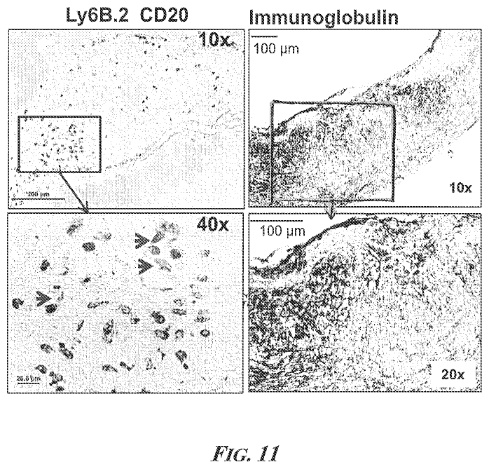

Belimumab, an anti-BAFF Ab approved by the US Food and Drug Administration completely blocks 3mer activity, but partly inhibits BAFF 60mer activity³⁴, and is mildly effective in lupus patients. Belimumab depletes mature B cells in humans. Binding of BAFF to the BR3 receptor plays a role in B cells survival Therefore, Belimumab treatment leads to depletion of mature B cells apart from inhibiting 60mer formation. The presently disclosed subject matter provides an antibody against the DE loop of BAFF (anti-DE3 Ab) and it was found that this Ab suppresses activation of B2 cells (B2 cells=FO+MZ B cells) without depletion of mature B cells (FIGS. 9 and 10). Therefore, it is tested whether inhibition of 60mer formation suppresses neutrophil mediated B2 cell activation and attenuate AAA formation.

### Example 6

### Genetic or Pharmacological Depletion of BAFF Attenuates AAA Formation

B cell expressing BAFF receptors have been found (FIG. 1), and neutrophils⁷ have been found in human and murine AAAs. Activated neutrophils secrete BAFF, which plays a role in B cell survival, activation and differentiation in to antibody producing plasma cells. Two BAFF neutralizing drugs are available for treatment of lupus patients; they are Belimumab (an anti-BAFF Ab) and Atacicept (a protein antagonist that inhibits BAFF and APRIL-mediated B cell survival and activation). While it is not known if patients receiving Belimumab or Atacicept develop AAA, the present studies demonstrate that injecting mice with an anti-BAFF Ab (binds to both the 3mer and 60mer BAFF), genetic depletion of BAFF (i.e. BAFF^{-/-}, not shown) or blocking BR3 receptor using anti-BR3 Ab strongly attenuates AAA formation (50-60% decrease in size of aortic diameter) (FIGS. 2A-2C).

### Example 7

### BAFF 60Mer as a Therapeutic Target

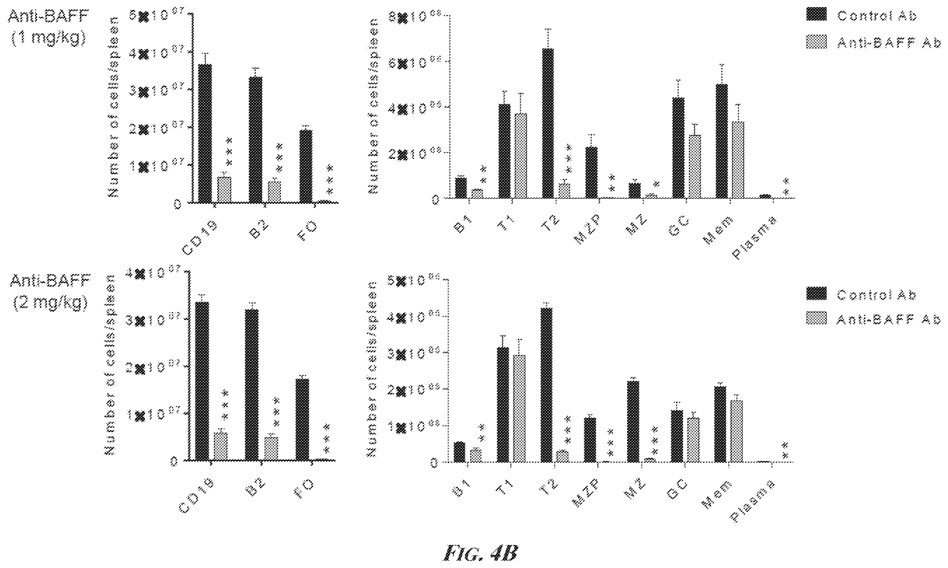

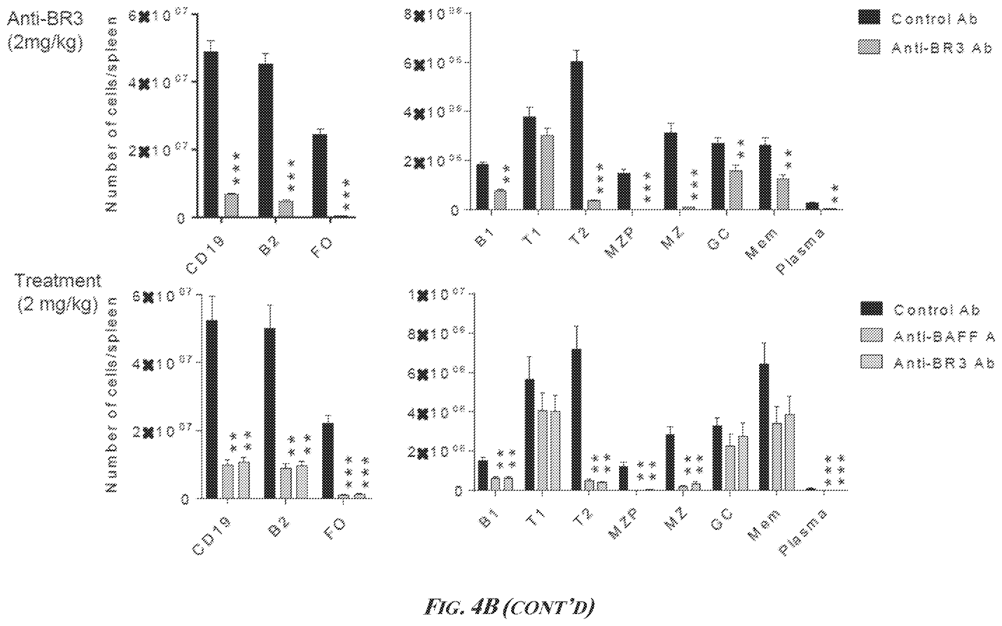

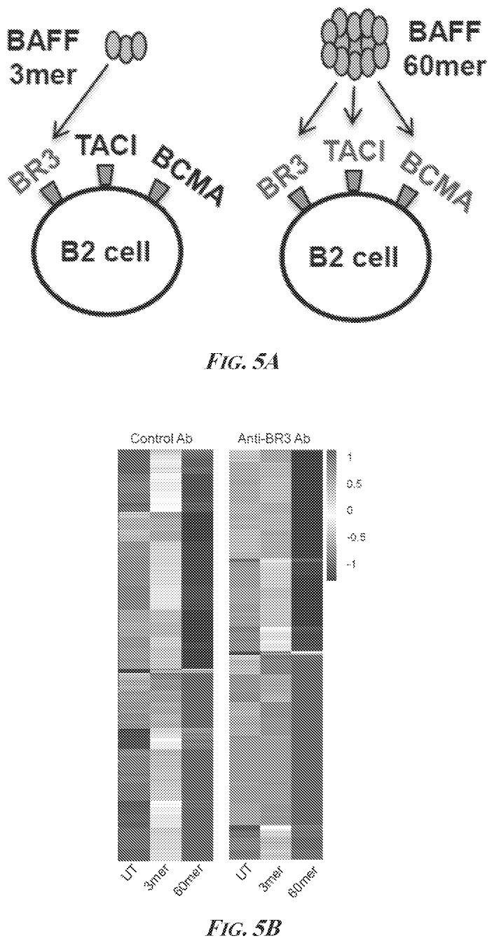

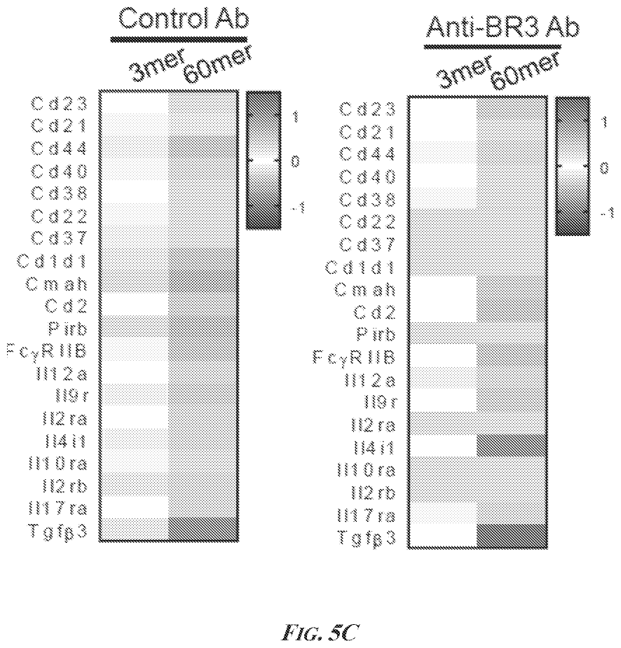

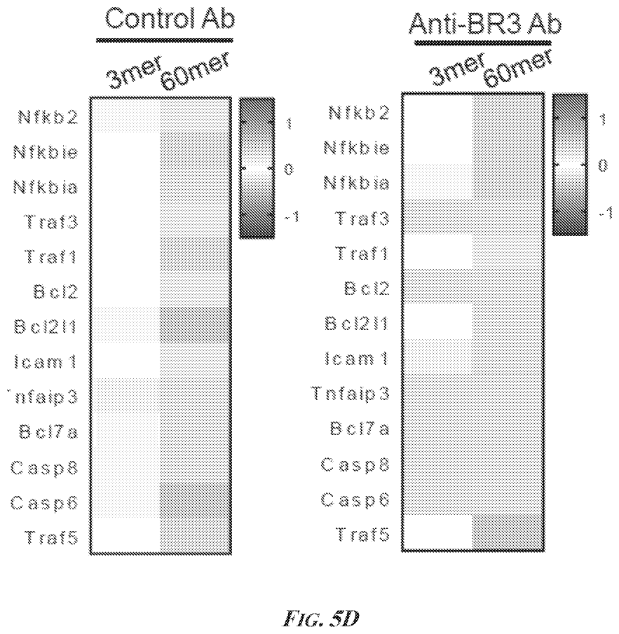

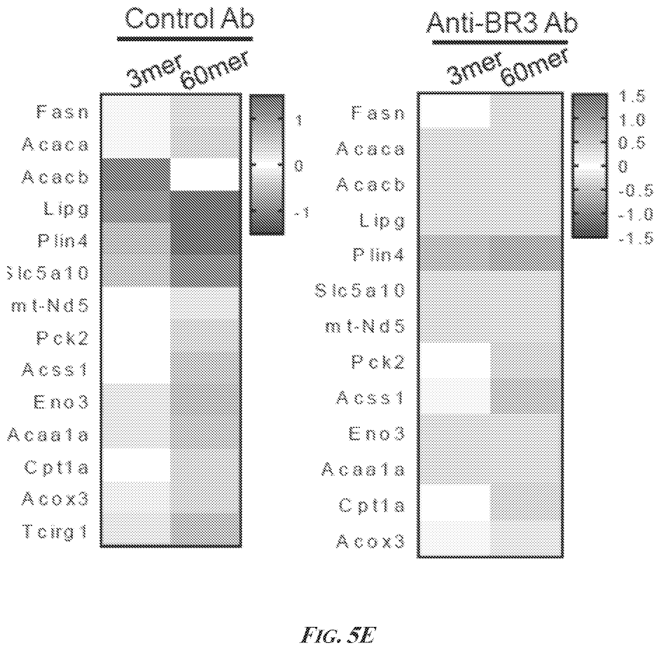

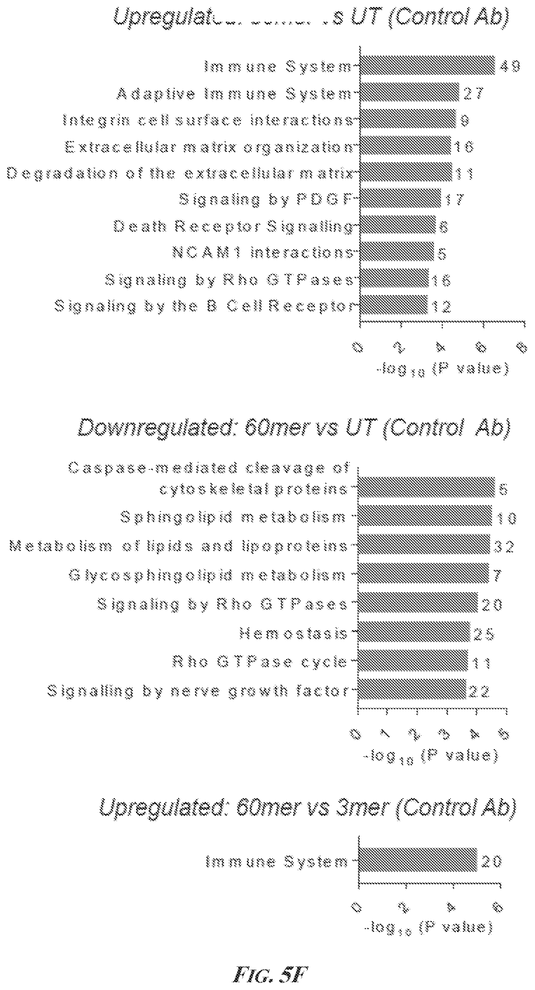

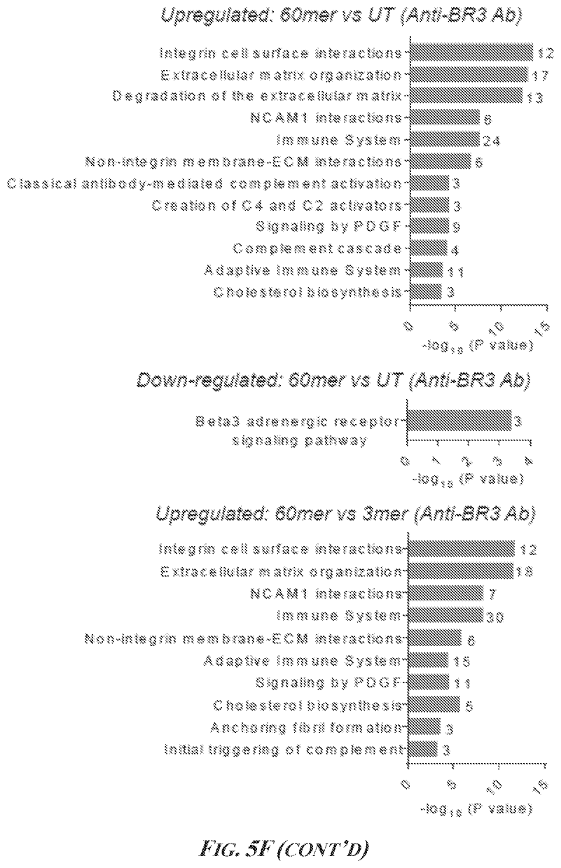

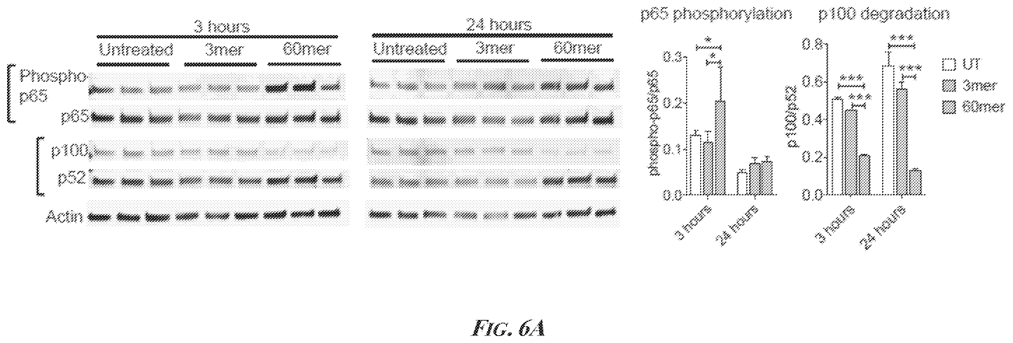

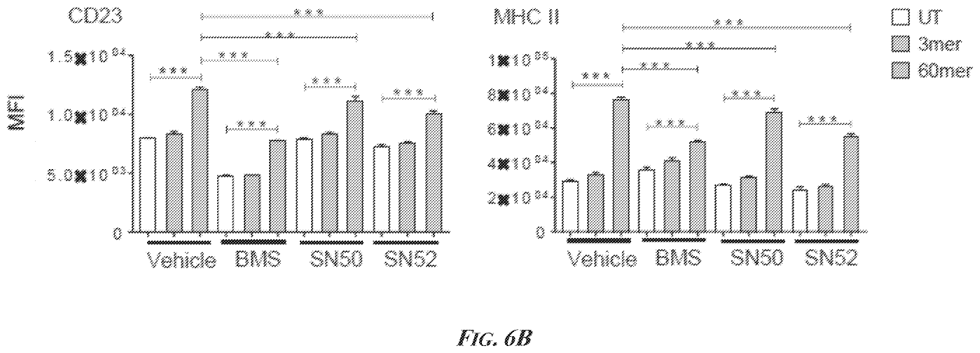

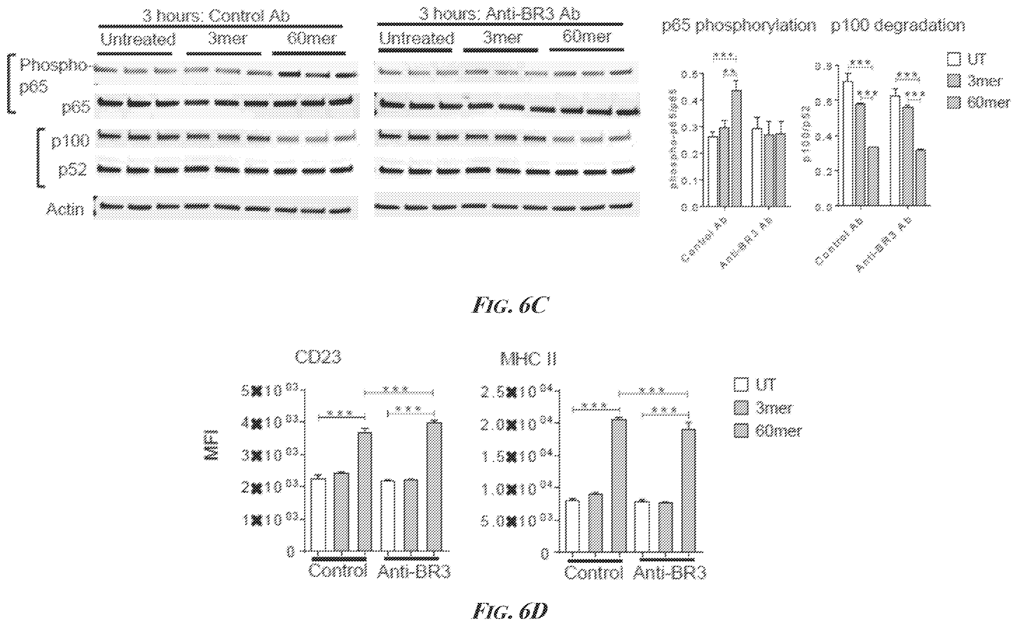

Depletion of neutrophils or B cells is not an attractive strategy to treat AAA as both of the cell types are required for host defense against infections. BAFF plays a role in survival, activation and differentiation of B cells. Therefore, Belimumab or anti-BAFF antibody treatment depletes mature B cell populations which include the memory (Mem) and plasma B cells (FIGS. 4A-4B). Therefore, it is pertinent to investigate how to suppress B cell activation without affecting the survival of B cells. BAFF 3mer promotes proliferation of activated B cells and the 60mer does to a greater extent¹². A similar trend was found in the potency of 3mer and 60mer (low and high, respectively) in activating NF-kB2 signaling (Western blot, FIG. 6A) and expression of B cell activation markers (Row data, FIG. 6B), and RNA-seg data (FIG. 5A-5D). Thus, BAFF 60mer is a therapeutic target for drug development against AAA. While it is not desired to be bound by any particular theory of operation, it is believed that inhibition of 60mer formation will allow naïve B cells to survive and proliferate but will not allow activation (which is required for differentiation in to antibody producing plasma cells). Since the BAFF is implicated in cardiovascular, metabolic and autoimmune diseases, the understanding the role of BAFF 60mer and the use of antibodies inhibiting 60mer formation has broader impact.

### Example 8

### Role of BAFF in Neutrophil-B Cell Crosstalk and AAA Formation

Neutrophils, upon activation by M-CSF and GM-CSF, secrete BAFF¹⁶, a factor required for B cell differentiation and survival. The presently disclosed data suggest that BAFF can exist as a 3mer or as a 60mer, and the 60mer is more potent in activating B cells than the 3mer. Furthermore, (i) B2 cells and plasma B cells (CD138+, Immunoglobulin+, but do not express conventional B2 cell markers such as CD19, B220 and CD20) are present in AAAs (FIG. 11), (ii) neutrophils localize close to B cells, and (iii) neutrophil activating cytokines G-SCF and GM-CSF are expressed in murine AAAs. These results together suggest possible interaction of activated neutrophils with B cells. Characterization of these infiltrated cells by flow cytometry has some limitations as it was previously found that enzymatic cocktail used for the digestion of aorta, digests some of the markers expressed on cell surface, particularly, CD23⁵. Therefore, the functional heterogeneity of infiltrated B cells and neutrophils in AAAs is determined by single-cell RNA-seq (Experiment 1a). Data demonstrate that similar to the BAFF depletion study, BAFF^{-/-} mice are protected from AAA formation. To determine if BAFF 3mer or 60mer is required for AAA formation, recombinant BAFF 3mer or BAFF 60mer are injected to BAFF^{-/-} mice and B cell activation, IgG1 and IgG2 production, aortic inflammation and experimental AAA formation is examined. (Experiment 1 b.1). To examine the roles of BR3 and TACI receptors on B cells in AAA formation in response to BAFF 60mer, BR3−/− or TACI−/− bone marrow is adoptively transplanted to irradiated BAFF−/− mice, BAFF 60mer is injected and experimental AAA is induced (Experiment 1b.2). To determine if BAFF produced from neutrophils is required for B cell activation and AAA formation, neutrophils isolated from WT mice are adoptively transferred to BAFF^{-/-} mice and AAA is induced (Experiment 1.4). Finally, to determine the role of IgG1 and IgG2 in FcγRIII (CD16)-mediated aortic inflammation and AAA formation, BAFF^{-/-} and CD16^{-/-}BAFF^{-/-} mice are injected with mouse IgG1 or IgG2 and AAA will be induced (Experiment 1.5).

Depletion of BAFF or blocking BR3 strongly attenuates AAA formation. To deplete BAFF, we used anti-BAFF Ab Sandy-2 (Adipogen) and to block BR3, we used anti-BR3 Ab from Biogen Idea USA. C57BL6 (WT) mice were injected with 1 or 2 mg/kg of anti-BAFF, 2 mg/kg of anti-BR3 Ab or a control Ab. After 14 days, the mice were injected again with the Abs and AAA was induced via topical elastase model. AAA formation and B cell phenotypes were determined after 14 days of AAA induction. Both of the antibodies depleted mature B cell populations in spleen and blood, and at 2 mg/kg dose significantly attenuated AAA formation in mice (FIGS. 2A-2C and 4A-4B), A similar phenotype of depletion of B cells and attenuation of AAA formation was observed in BAFF−/− mice (not shown). Along the plasma immunoglobulins, the level of IgG1, IgG2a and IgG2b were significantly lower in the BAFF depleted and BR3 blocked mice. Interestingly, BAFF depletion did not affect neutrophil infiltration (Ly6b.2) and FcγRIII expression, but reduced IgG accumulation in aorta. These results suggest a critical role of pathogenic IgG deposition in BAFF-mediated AAA formation.

The 60mer induces both NF-kB1 and -kB2 signaling and increases CD23 and MHCII expression on B cells. Prior to the instant disclosure, it is not clear if BAFF 3mer and 60mer differentially activate NF-kB signaling in B cells. Therefore, total splenic B cells from C57BL/6 male mice were isolated by using pan B cell isolation kit from Miltenyi Biotec (Cat #130-095-813) and treated 6 million cells with 100 ng/ml of human recombinant BAFF 3mer (Flag-tagged BAFF, AdipoGen) and 60mer (His-tagged BAFF, AdipoGen) for 3 hours or 24 hours, or left untreated, and analyzed NE-kB signaling from the whole cell extracts using Western blot (FIG. 6A). After 3 hours of treatment, the 60mer significantly activated NF-kB1, which was diminished by 24 hours, whereas, no significant effect was found by the 3mer. Both the 3mer and 60mer activated NF-kB2 signaling at 3-hour and 24-hour time points, though the 60mer response was stronger than the 3mer. Next, to determine if BAFF treatment induces NF-kB-dependent B cell activation, splenic B cells were pretreated with BMS 345541, SN50 or SN52 (inhibits both NF-kB1 and -kB2, NF-kB1 or NF-kB2-mediated gene transcription, respectively) for 1 hour, before treating with BAFF 3mer and 60mer. After 24 hours, expression level of CD23 and MHC H was determined by flow cytometry (FIG. 6B). Interestingly, compared to the untreated and the 3mer, the 60mer significantly increased CD23 and MHC H expression, which was suppressed by BMS and SN52, and to a lesser extent by SN50. Altogether, these results suggest that BAFF 60mer promotes B cell activation by utilizing both NF-kB1 and -kB2 signaling. 60mer activates synthesis of genes required for B cell activation and survival. Prior to the instant disclosure, it was unknown if 3mer and 60mer differentially regulate gene synthesis in B cells. Therefore, splenic total B cells with 100 ng/ml of BAFF 3mer, 60mer or left untreated in triplicates for 4 hours, isolated total RNA and performed RNA sequencing (RNA-Seq) by Novogene Corporation (Wilmington, Del., USA). Novogene performed the RNA sequencing in Illumina Hiseq 4000 following the state of the arts techniques (FIGS. 5A-5E). Importantly, the Pearson coefficient, R², between samples of a treatment was >0.92 suggesting very low variability. It was found that, among the untreated, 3mer and 60mer treated B cells, 62 genes were uniquely expressed by the 3mer, whereas, 276 genes are uniquely expressed by the 60mer. Relative to the untreated and the 3mer, 60mer significantly upregulated synthesis of genes involved in B cell activation (CD21, CD23, 9R CD40 and MHCII), NF-kB signaling (NFkb2, Nfkbie, Nfkbia, Traf1, and Traf3), anti-apoptosis (Bcl2, Bcl2l1), cell division and cancer (FIGS. 5C-5D). Interestingly, (1) no known inflammatory interleukins were found and (2) expression level of all 3 of the negative co-receptors for B cell receptor activation⁴⁶ such as IgG receptor FcγRIIB (CD32b), CD22 and PirB was lower, suggesting the cells are in an activated state. We further identified S1P1 (sphingosine-1-phosphate receptor 1 helps in B cell migration⁴⁷) as a novel BAFF 60mer-suppressed gene. We found BAFF 60mer significantly suppressed the surface expression of S1 P1, which was not affected by NF-kB signaling. Altogether, these results suggest that, the 60mer uniquely marks B cells towards an activated and proinflammatory phenotype.

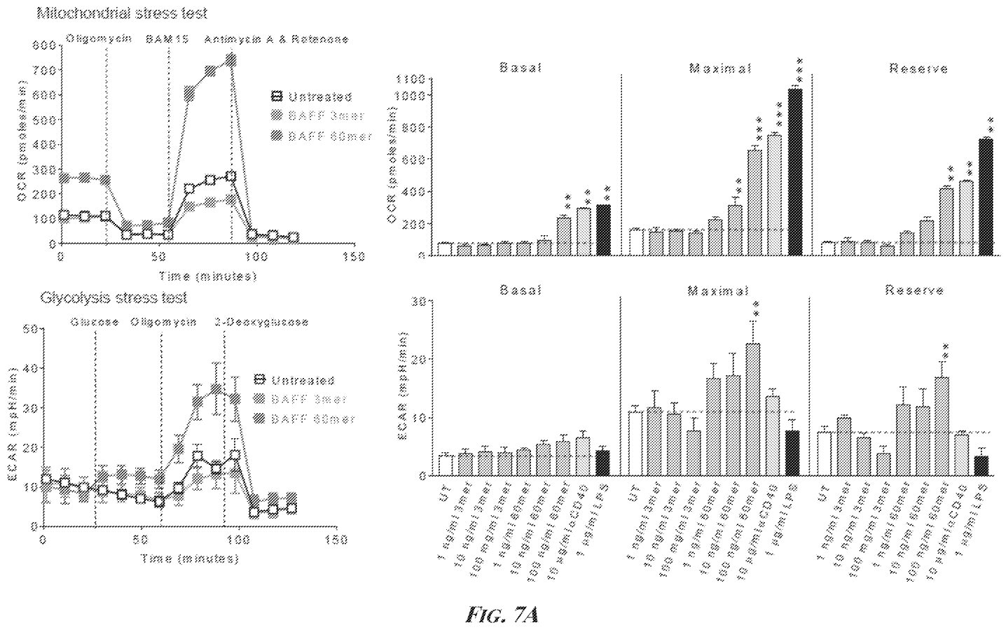

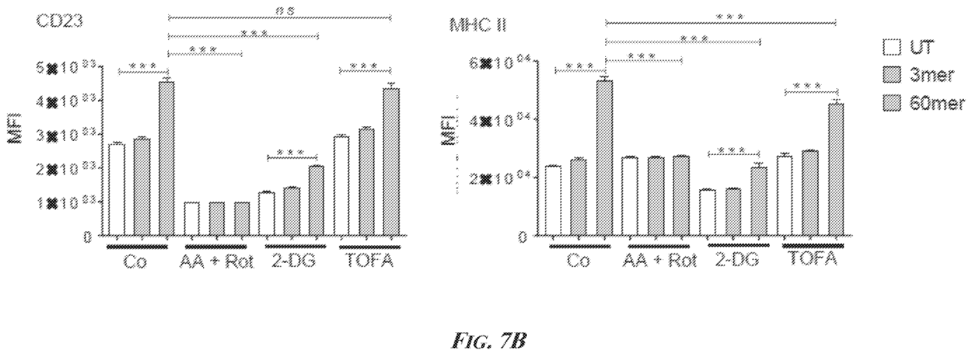

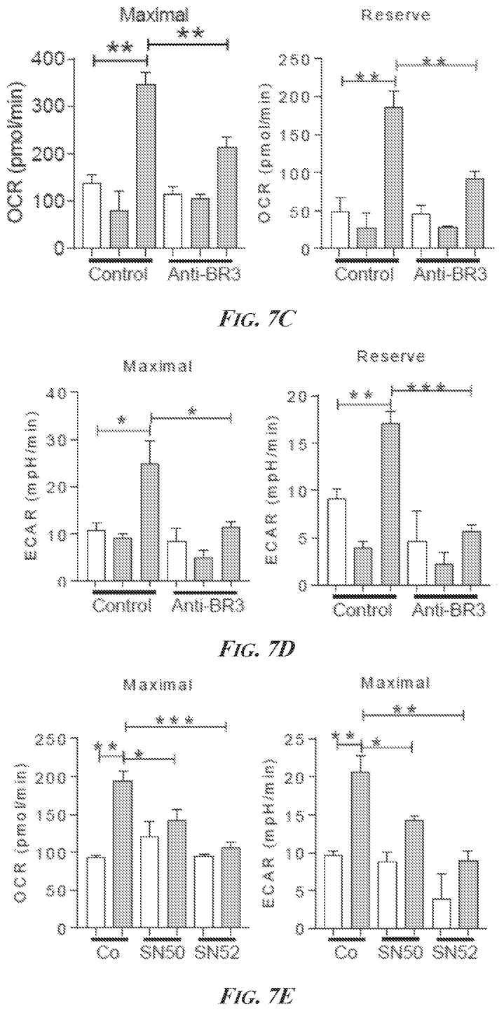

BAFF 60mer significantly increases cellular glycolysis and mitochondrial respiration. Seahorse XF Cell Mito Stress Test and Glycolysis Stress Test were used to measure mitochondrial respiration and glycolysis; respectively. Total B cells isolated from spleens of WT mice were seeded at 1 million cells/well in Seahorse XF 24-well microplate coated with polylysine and left untreated or treated with 1, 10 and 100 ng/ml of BAFF 3mer or 60mer, 10 μg/ml anti-CD40 Ab (positive control for NF-kB2 signaling⁴⁸) and 1 μg/ml of LPS (positive control for NF-kB1 signaling) for 24 hours. After the incubation, the assays were performed separately using Seahorse XF24 Extracellular Flux Analyzer. In the mitochondrial stress test different drugs were added sequentially to specifically regulate electron transport through the mitochondrial electron transport chain. The electron transport affects cellular O2 consumption rate (OCR) which is recorded by the Seahorse. During glycolysis, glucose in the cell is converted to pyruvate, and then to lactate leading to extrusion of protons into the extracellular medium. The rate of release of protons is measured as extracellular acidification rate (ECAR) The results demonstrate increasing concentration of BAFF 60mer increases mitochondrial respiration and glycolysis compared the 3mer and untreated B cells (FIGS. 7A-7C). Interestingly, anti-CD40 Ab and LPS significantly increased OCR, but not ECAR suggesting that the BAFF 60mer uniquely reprogram energy metabolism in B cells. Furthermore, blocking BR3 receptor by anti-BR3 Ab significantly attenuated BAFF 60mer-mediated increase in OCR and ECAR, suggesting a critical role of BR3 in BAFF 60mer-mediated metabolic remodeling in B cells.

Experiment 1a: Determine the functional heterogeneity of infiltrated B cells and neutrophils in murine AAAs by single cell RNA-seq. Live neutrophils (at days 3 and 7 after AAA induction⁷) and B cells (at days 7 and 14 after AAA inductions) from murine AAA (after enzymatically digestion) and blood (as a negative control) are labeled with fluorescent-conjugated antibodies, sort-purified and submitted in University of Virginia core facility for performing single cell RNA sequencing and subsequent data analysis. Neutrophils are identified as CD45+CD11b+Ly6G+, B cells as CD45+CD19+ and plasma cells as CD45+CD138+CD19−CD3−F4/80−.

˜95% pure cells are typically observed in preparations. ˜3×10⁴ neutrophils and ˜9×10³ B cells are present/mouse AAA at day 7 after aneurysm induction by elastase perfusion model^{5,7}. Therefore, cells from 4 AAAs are pulled per sample of neutrophil, B cell and plasma cell. Using unbiased hierarchical clustering, we expect to find (i) >95% of the neutrophils as B cell-helper neutrophils (express low level of CD15 and CD16) and the remaining conventional neutrophils (express high level of CD15 and CD16, and (ii) multiple populations of B cells. BAFF expression level are expected higher in the B cell-helper neutrophil population. The major population of B cell subset would be plasma cells (express BCMA), followed by follicular B cells (express BR3 and TACI) showing signature of being stimulated by BAFF 60mer such as expression of genes involved in NF-kB2 signaling, B cell activation markers, and, suppression of S1P1.

Experiment 1 b.1: Determine if BAFF 60mer promotes AAA formation, Endotoxin free purified human BAFF 60mer (aa134-285) and 3mer (aa134-285 with His218Ala) has been prepared from the supernatant of HEK293T cells transfected with an expression plasm id pUNO bearing BAFF expressing plasmids. Inject 100 μg of 3mer or 60mer in to male or female BAFF^{-/-} mice, and determine stability of BAFF after 6, 12, 24, 48, and 72 hours, and, at the same time points, quantify BAFF by ELISA. After optimizing the doses, BAFF^{-/-} mice are injected with the 3mer, 60mer or saline only, AAA is induced by topical elastase method and aneurysm growth is determined after 14 days. B cell subtypes and activation markers are determined from AAA tissue, spleen, peritoneal fluid, blood and bone marrow. Plasma BAFF and immunoglobulins are quantified. Activation of B cells isolated from spleens and aortas of BAFF 60mer injected mice is determined by increased NF-kB1 and NF-kB2 signaling, increased MHCII and decreased S1P1, and, increase in both glycolysis and mitochondrial respiration. Similarly, BAFF 3mer and 60mer is injected to ApoE^{-/-}BAFF^{-/-} mice and AAA formation is determined using AngII infusion model.

The injected BAFF 3mer and 60mer are detected till 24 hours by Western blot, and for 48 hours by ELISA. It is expected that injection of 3mer to the BAFF^{-/-} mice replenishes all of the B cell subtypes, plasma IgG1 and IgG2 subtypes and AAA growth similar to the WT (C57BL/6) mice. The 60mer injected BAFF^{-/-} and ApoE^{-/-}BAFF^{-/-} mice do not only replenish B cells, but also increase the number of B2 cells more than age matched WT mice and activate B cells to produce more IgG1 and IgG2. The 60mer treatment is expected make AAA size larger than the 3mer. In the AngII infusion model, an increase in AAA rupture in mice receiving BAFF 60mer is expected.

Experiment 1b.2: Examine the roles of BAFF receptors on B cells in AAA formation in response to BAFF 60mer. WT, BAFF^{-/-}, BR3^{-/-} or TACI^{-/-} bone marrow cells are adoptively transferred to irradiated BAFF−/− mice, BAFF 3mer or 60mer are injected and AAA is induced. Plasma cells primarily express the BAFF receptor BCMA and BAFF 60mer promotes plasma cell survival, therefore, BCMA^{-/-} mice are included in this experiment. Since B2 cells express both BR3 and TACI receptors, it is expected that 60mer injection to BAFF−/− mice receiving WT or BAFF−/− bone marrow cells will form AAA. BAFF−/− mice receiving BR3^{-/-}, TACI^{-/-} or BCMA^{-/-} bone marrows will have impaired development in B2 and plasma cells and will not form AAA.

Experiment 1c: Determine if BAFF secreted by neutrophils is required for B cell activation and AAA formation. BAFF is ubiquitously expressed; therefore, to determine the role of neutrophil-secreted BAFF in B cell activation and AAA formation, neutrophils isolated from bone marrows of WT mice are adoptively transferred to BAFF mice and examine AAA formation, accumulation of BAFF+ neutrophils in AAA and activation of aortic infiltrated B cells. Because of technical limitation to identify BAFF 3mer and 60mer in AAAs, neutrophils from AAAs of WT mice are isolated after 7 days of aneurysm induction and perform following experiment: directly co-culture aortic neutrophils or neutrophils isolated blood (as a control) with mouse splenic B cells and quantify the expression of MHCII and S1P1 on B cell surface by flow cytometry and quantify the nuclear translocation of phospho-p65 and p52 using Amnis ImageStreamX Mark H (imaging flow cytometry). An increase in AAA formation and accumulation of BAFF+ neutrophils is expected AAAs BAFF^{-/-} mice adoptively transferred with WT neutrophils, Results demonstrate that BAFF 60mer, but not the 3mer, induces expression of MHCII and decreases the expression of S1 P1. Therefore, in the co-cultures, B cells cultured with aortic neutrophils will demonstrate increased expression of MHCII and decreased expression of S1P1, and increased number localization of phospho-p65 and p52 compared to bone marrow neutrophils.

Experiment 1d: Determine the role of IgG1 and IgG2 in FcγRIII (CD16)-mediated aortic inflammation and AAA formation. For this experiment, FcγRIII^{-/-} mice (86.129P2-Fcgr3tm1 Jsv/2J, id. 9637) from Jackson laboratories were breed with BAFF^{-/-} mice to obtain FcγRIII^{-/-}BAFF^{-/-} mice, BAFF^{-/-} and FcγRIII^{-/-} BAFF^{-/-} mice are injected with mouse IgG1 or IgG2 and AAA is induced. Attenuated AAA formation is expected in these mice. Administering IgG1 or IgG2 will lead to deposition of these antibodies in aorta of BAFF^{-/-} mice, increased aortic inflammation (as determined by overexpression of proinflammatory genes), increased MMP production (by zymogram), degradation of elastin layers, and loss of smooth muscle α-actin staining. However, BAFF^{-/-} FcγRIII^{-/-} mice receiving IgG1 or IgG2 will demonstrate attenuated AAA.

### Example 9

### Inhibition of BAFF 60Mer Formation Attenuates Neutrophil-B Cell Inflammatory Crosstalk, B2 Cell Activation and AAA Formation

The currently available BAFF depletion biologics Belimumab and Atacicept deplete mature B cell populations. This is undesirable because the depleted mature B cell populations include memory B cells, which are required for body's defense against an infection. Based on the presently disclosed data on the role of BAFF 60mer on B cell activation (FIGS. 5A-7E), a polyclonal Ab was developed against the DE loop BAFF (anti-DE3 Ab). It was found that this Ab suppresses activation of B cells without depletion. The anti-DE3 Ab is polyclonal Ab; therefore, monoclonal anti-DE Abs are developed, which will be highly specific and reproducible in activity. These antibodies are validated using biochemical and biophysical methods (Experiment 2a). To develop an intervention strategy, it is examined if the anti-DE Abs inhibit the growth of established murine AAAs (Experiment 2b). In order to translate findings to a human setting, the effectiveness of anti-DE Abs is compared with available anti-BAFF biologics in attenuating activation of B cells in human neutrophil-B cell co-cultures. (Experiment 2c). See also Example 10.

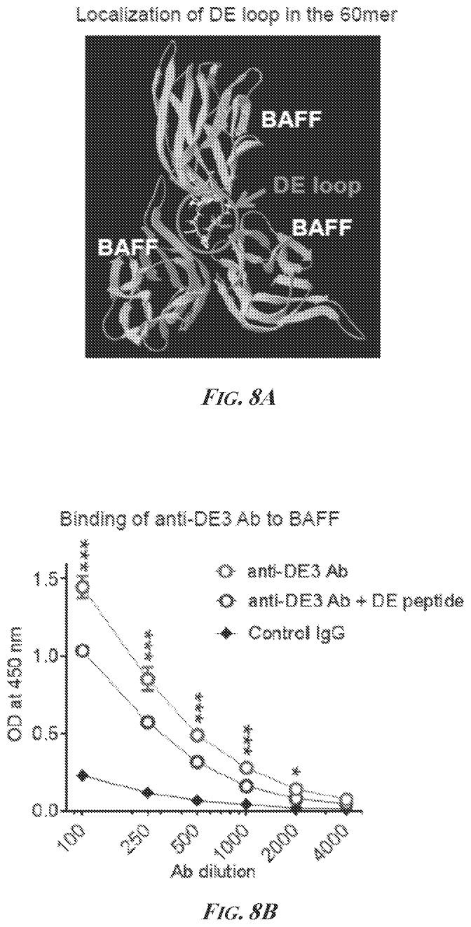

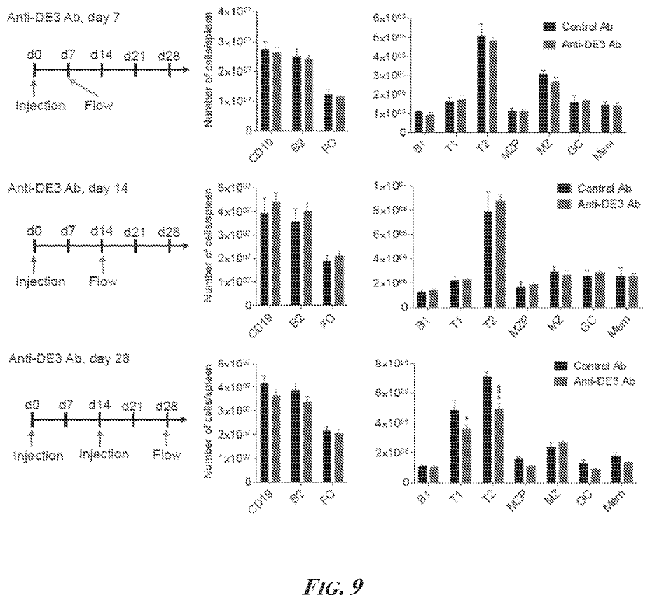

One of the presently disclosed anti-DE Ab, anti-DE3, binds to BAFF in vitro, and suppresses CD23 expression in B cells in a B cell: neutrophil co-culture model. The anti-DE3 Ab was raised against a peptide (KVHVFGDELSLVTC, SEQ ID NO: 2) encompassing DE loop (required for 60mer formation) of BAFF (FIG. 8B) in rabbit via GenScript (NJ, USA). To determine if the anti-DE3 Ab binds to DE loop of BAFF, we added various dilutions of anti-DE3 Ab, anti-DE3 Ab with the DE peptide, and a rabbit IgG, to BAFF 3mer coated on an ELISA plate. The bound antibodies were detected by HRP-conjugated goat anti-rabbit Ab and Pierce TMB Substrate Kit (ThermoFisher Scientific). The results demonstrate that the DE peptide significantly reduced the binding of the anti-DE3 Ab with BAFF (FIG. 8B).

Anti-DE3 Ab suppresses expression of B cell activation markers without depleting B cells. Anti-DE3 Ab (4 mg/kg) or a control Ab was injected to C57BL16 male mice and B cell sub-types were quantified after 7, 14 or 28 days of injection. In the 28-day group, a total of two injections were given, i.e. on day 0 and day 14. Compared to anti-BAFF Ab study (FIGS. 4A-4C), injection of two doses of anti-DE3 Ab (28-day group) only partly depleted T1 and T2 B cells and significantly attenuated surface expression of B2 cell marker CD23 and B cell activation marker MHCII in follicular B cells present in spleen and blood (FIGS. 9 and 10). No significant differences in the number of B cell sub-types or surface expression of CD23 and MHCII were observed in 7 and 14-day groups.

Experiment 2a: Testing whether the antibody targeted against homogenization site of BAFF 3mer inhibits 60mer formation. Mouse monoclonal antibodies are generated against the DE loop of BAFF in accordance with techniques described herein by using The Antibody Engineering and Technology at the University of Virginia, Charlottesville, Va., United States of America. For biophysical analysis, proteins in milligrams are produced in Escherichia coli (E. coli). Human soluble BAFF 60mer (aa134-285) and the 3mer (H218A aa134-285) is prepared in E. coli expression plasmid pReceiver and purifying the protein using Q-Sepharose and S-Sepharose. 15N (Nitrogen)-labeled BAFF 3mer is also prepared. Heteronuclear single quantum coherence (HSQC) spectra of 1 mM BAFF 3mer is recorded in presence or absence of monoclonal anti-DE Ab or control Ab on a 600 MHz NMR spectrometer at UVA. Anti-DE Ab binding sites on BAFF are identified by changes in the chemical shifts of peaks in the 15N-1H HSQC spectra. Taking advantage of existence of soluble BAFF (aa134-285) as 3mer at pH 6, and 60mer at pH 7.4, anti-DE Ab or control Ab is added to 15N-labeled BAFF at pH 6, and dialyze against pH 7.4 buffer, and record HSQC spectra to determine if the Anti-DE Ab binds to BAFF 3mer and inhibits 60mer formation. It is tested if the monoclonal anti-DE Ab lose binding to BAFF if the BAFF DE loop mutant ‘BAFF His218Ala’ (aa134-285) is used, in a similar ELISA method as described elsewhere herein. The binding constant of the antibody using fortéBIO Octet RED96 available at the University of Virginia, Charlottesville, Va., United States of America, Thus, anti-DE Ab is rigorously validated. A significant shift in peaks of amino acids of DE loop in the 15N-1H HSQC spectra of BAFF is expected after binding of anti-DE monoclonal Ab. This ELISA is expected to reveal that the anti-DE Ab completely lose binding to H218A BAFF, A high binding constant (>10⁸) of the anti-DE Ab to FLAG-tagged BAFF 3mer is also expected.

Experiment 2b: Examine if the anti-DE Abs suppress the growth of established murine AAAs. 28-day angiotensin II (AngII) infusion (1,000 ng/kg/rain) model is used in AAA in ApoE−/− mice. 14 days after AngII infusion, AAA size is determined by ultrasound imaging of the live mice. Thereafter, 4 mg/kg (˜100 μg) of the monoclonal anti-DE Ab or a control Ab is injected on days 14 and 21, and AAA size is determined in day 28 using ultrasound imaging. The mice are sacrificed, and B cells activation is examined in spleen and AAA and IgGs are quantified by ELISA. It is expected that the anti-DE Ab treatment will significantly attenuate the growth of AAA and decrease AAA rupture rate with a concomitant decrease in B2 cell activation and IgG1, IgG2a and IgG2b Ab production without depletion of mature B cell populations.

Experiment 2c: Determine if activated neutrophils stimulate human B cells, which can be attenuated by the anti-DE Abs. Human promyeloblast cells HL-60 (ATCC CCL-240) cells are differentiated into neutrophils using DMSO⁵³. CD19+ B cells are isolated using MACS columns (Miltenyi Biotec) from human peripheral blood mononuclear cells (PBMC) obtained from Lonza, USA. HL-60 neutrophils are transfected with SMARTpool: Accell TNFRSF13C (BAFF) siRNA or a scrambled siRNA from Dharmacon, activated by G-CSF and GM-CSF treatment; washed and co-cultured with B cells using trans-wells. Activation of B cells in the co-cultures are examined by flow cytometry and Seahorse Extracellular Flux Analyzer. Next, B cells are co-cultured with HL-60 neutrophils in presence of a control Ab, anti-DE Ab; Belimumab or Atacicept and activation of B cell is examined. >90% knock-down of BAFF gene is expected, as determined by real-time RT-PCR. The knock-down is further verified using Western blot of whole cell lysate and ELISA of culture supernatants after G-CSF and GM-CSF treatment. B cells co-cultured with BAFF deficient neutrophils are expected to demonstrate attenuated activation compared to BAFF sufficient neutrophils. It is also expected that that the anti-DE Ab will significantly attenuate neutrophil-mediated B cells activation but only the anti-DE Abs will not affect the count of viable B cells.

### Example 10

This Example tests whether novel reagents block DE loop inhibit BAFF 60mer formation and reduce plasma B cell differentiation. Isolated mouse bone marrow neutrophils are stimulated with GM-CSF to secrete BAFF and co-cultured with B cells in presence or absence of anti-DE loop antibody or peptide, and B cell phenotype is determined.

In mouse plasma, only 4% of the BAFF exists as 60mer. The BAFF 60mer is primarily formed by multimerization of BAFF 3mers via the solvent accessible loop of BAFF. The residue histidine 218 (H218) in the DE loop is critical for 60mer formation, as mutation of H218 to alanine abrogates 60mer formation, but retains 3mer formation and binding of BAFF to BAFF receptor BR37. Moreover, BAFF 60mer is highly active than the 3mer. Therefore, in some embodiments, approaches design reagents that block solvent accessibility of H218, and hence inhibit BAFF 60mer formation.

Development of native PAGE method to determine BAFF multimerization: Samples (purified proteins and culture supernatants) were mixed with glycerol (final concentration 10%) and proteins are separated via running on NativePAGE™ Novex™ 4-16% Bis-Tris Protein Gel with NativePAGE™ Running Buffer and NativeMark™ Unstained Protein Standard (ThermoFisher Scientific). Recombinant BAFF 3mer and 60mers (from AdipoGen) were used as controls, Subsequently, Western blot was performed against Anti-BAFF Antibody (A316530, EMD Millipore) and LI-COR secondary antibody, and scanned using a LI-COR instrument. In the blot, BAFF 60mer is identified as 1020 & 720 kDa band and numerous lower molecular weight multimers, and 3mer is identified as 51 kDa band. Interestingly, the BAFF antibody bound strongly to BAFF 60mer compared to the BAFF 3mer.

The DE loop amino acid sequence in BAFF is ‘KVHVFGDELS’ (SEQ ID NO: 1), Based on immunogenicity required for antibody generation, the sequence ‘KVHVFGDELSLVT’ (SEQ ID NO: 2) sequence was selected. Rabbits were immunized with peptide-KLH conjugates and the antibodies were affinity purified against the peptide. Titer of the antibody was found to be 1:128,000. Immunohistochemistry on AAA tissue collected from 14 days after AngII infusion in ApoE−/− mice demonstrated co-localization of neutrophils and B cells, and GM-CSF producing cells. By way of additional example, polyclonal Abs are generated against multiple lengths comprising DE loop and neighboring amino acids such as: LIQRKKVHVFGDELSLVTLF (SEQ ID NO: 3); IQRKKVHVFGDELSLVTL (SEQ ID NO: 4); KKVHVFGDELSL (SEQ ID NO: 5); LIQRKKVHVFGDELS (SEQ ID NO: 6); LIQRKKVHVFGDELSL (SEQ ID NO: 7); IQRKKVHVFGDELSLV (SEQ ID NO: 8); QRKKVHVFGDELSLVT (SEQ ID NO: 9); RKKVHVFGDELSLVTL (SEQ ID NO: 10); and LIQRKKVHVFGD (SEQ ID NO: 11). Bold indicates the DE loop amino acids.

Mouse monoclonal antibodies are be produced via hybridoma technology. Briefly, the DE loop peptide (10 amino acids: KVHVFGDELS, SEQ ID NO: 1) is synthesized in Anaspec and monoclonal antibodies are synthesized and validated by Antibody Engineering and Technology Core at the University of Virginia, Charlottesville, Va., United States of America By way of additional example, monoclonal Abs are generated against multiple lengths comprising DE loop and neighboring amino acids such as: LIQRKKVHVFGDELSLVTLF (SEQ ID NO: 3); IQRKKVHVFGDELSLVTL (SEQ ID NO: 4); KKVHVFGDELSL (SEQ ID NO: 5); LIQRKKVHVFGDELS (SEQ ID NO: 6); LIQRKKVHVFGDELSL (SEQ ID NO: 7); IQRKKVHVFGDELSLV (SEQ ID NO: 8); QRKKVHVFGDELSLVT (SEQ ID NO: 9); RKKVHVFGDELSLVTL (SEQ ID NO: 10); and LIQRKKVHVFGD (SEQ ID NO: 11). Bold indicates the DE loop amino acids.

DE loop mimetic peptides are also designed. BAFF monomers adapt a tumor necrosis factor (TNF)-like jellyroll fold. It comprises five antiparallel 3-sheets (A-E) arranged in a Greek-key motif. The loop between D and E antiparallel 6-strands is the DE loop which contains the H218 residues. The loop is 10 amino acid long and it is unstructured; therefore, it is not expected that addition of only the synthetic peptide mimicking the loop will inhibit BAFF 60mer formation, mainly because of lack of structure, and hence specificity. Therefore; multiple loop peptides are synthesized, which include 1, 2, 3; 4 or 5 residues from the D and E β-strands. To enhance stability and protect the peptide from proteases, 1, 2, 3-triazole ring are added. Altogether, macrocyclic peptides are generated, which have a higher possibility to interact with DE loop of BAFF 3mers and inhibit higher order multimerization.

A representative cyclic peptide is DE1akm, IQRKKVHVFGDELSLVTL (SEQ ID NO: 12), head to tail cyclization. The DE1 akm peptide is designed to retain a part of a and ‘E’ beta-sheets and the DE loop. This structure should serve as a competitive inhibitor. Another representative cyclic peptide is DE3akm, VHVFGDEL (SEQ ID NO: 13), head to tail cyclization. Another representative peptide is DE4akm, Ac-RKKVHVFGDELSLV-NH2 (SEQ ID NO: 14). These peptides were prepared by a synthetic route but can be prepared by a suitable approach as disclosed herein and as would be apparent to one of ordinary skill in the art up on a review of the instant disclosure as can suitable modified versions, substantially homologous amino acid sequences, fragments, and/or variants of these peptides. The term “DE loop mimetic peptide” thus includes the representative peptides sequences disclosed herein, suitable modified versions, substantially homologous amino acid sequences, fragments, and/or variants of these peptides.

In one approach for testing the compounds, secreted form of human BAFF (aa134-285) is expressed in HEK293 cells in presence of DE loop Ab or control IgG and multimerization of BAFF is determined from culture supernatant using a native PAGE and Western blotting method. Next, to mimic in vivo scenario, isolated mouse bone marrow neutrophils are stimulated with GM-CSF, co-cultured with B cells in presence or absence of DE loop antibody or peptide and, differentiation of B cells and NF-KB signaling is determined.

It is expected that the rabbit polyclonal Ab and mouse monoclonal Ab against DE loop will inhibit BAFF 60mer formation in BAFF-transfected HEK293T cell culture supernatant and neutrophil-B cell co-culture. In native PAGE it is expected to find anti-BAFF antibody detecting a band corresponding to the molecular weight of 3 BAFF+1 IgG=17 kDa×3+150 kDa=201 kDa. It is expected that the DE loop macrocyclic peptide containing 5 residues each from ‘D’ and ‘E’ β-strands will inhibit BAFF 60mer formation in culture supernatants. In case the macrocyclic peptide is found degraded, D-form of amino acids will be used for peptide synthesis, which is known to be protected from protease digestion. However, if D-forms are used, conformation of the peptide, which contains partial ‘D’ and ‘E’ β-strands, may change and may bind to the DE loop. Therefore, if D-forms are used, conformation of the peptide is determined using micro-crystallography methods. It is expected that both the DE loop Ab and peptide significantly inhibit neutrophil activated differentiation of B cells to plasma cell. Cell death is closely monitored in this experiment.

### REFERENCES CITED IN EXAMPLES

The disclosures of each and every patent, patent application, and publication cited herein are hereby incorporated by reference herein in their entirety.
1. Ailawadi G, Knipp B S, Lu G, Roelofs K J, Ford J W, Hannawa K K, Bishop K, Thanaporn P, Henke P K, Stanley J C and Upchurch G R, Jr. A nonintrinsic regional basis for increased infrarenal aortic MMP-9 expression and activity. Journal of vascular surgery. 2003; 37:1059-66.
2. Airhart N, Brownstein B H, Cobb J P, Schierding V V, Arif B, Ennis T L, Thompson R W and Cerci J A. Smooth muscle cells from abdominal aortic aneurysms are unique and can independently and synergistically degrade insoluble elastin. Journal of vascular surgery. 2014; 60:1033-41; discussion 1041-2.
3. Sinha I, Hannawa K K, Eason J L, Ailawadi G, Deogracias M P, Bethi S, Ford J W, Roelofs K J, Grigoryants V, Henke P K, Stanley J C and Upchurch G R, Jr, Early MT-1 MMP expression following elastase exposure is associated with increased cleaved MMP-2 activity in experimental rodent aortic aneurysms. Surgery, 2004; 136:176-82.
4. Woodrum D T, Ford J W, Ailawadi G, Pearce C G, Sinha I, Eagleton M J, Henke P K, Stanley J C and Upchurch G R. Jr. Gender differences in rat aortic smooth muscle cell matrix metalloproteinase-9. J Am Coll Sorg. 2005; 201:398-404.
5. Meher A K, Johnston W F, Lu G, Pope N H, Bhamidipati C M, Harmon D B, Su G, Zhao Y, McNamara C A, Upchurch G R, Jr. and Ailawadi G. B2 cells suppress experimental abdominal aortic aneurysms. The American journal of pathology. 2014; 184:3130-41.
6. Schaheen B, Downs E A, Serbulea V, Almenara C C, Spinosa M, Su G, Zhao Y, Srikakulapu P, Butts C, McNamara C A, Leitinger N, Upchurch G R, Jr., Meher A K and Ailawadi G. B-Cell Depletion Promotes Aortic Infiltration of Immunosuppressive Cells and Is Protective of Experimental Aortic Aneurysm. Arteriosclerosis, thrombosis, and vascular biology. 2016; 36; 2191-2202.
7. Meher A K, Spinosa M, Davis J P, Pope N, Laubach V E, Su G, Serbulea V, Leitinger N, Ailawadi G and Upchurch G R, Jr. Novel Role of IL (Interleukin)-1 beta in Neutrophil Extracellular Trap Formation and Abdominal Aortic Aneurysms. Arteriosclerosis, thrombosis, and vascular biology. 2018; 38:843-853.
8. Kyaw T, Tay C, Hosseini H, Kanellakis P, Gadowski T, MacKay F, Tipping P, Bobik A and Toh B H. Depletion of B2 but not B1a B cells in BAFF receptor-deficient ApoE mice attenuates atherosclerosis by potently ameliorating arterial inflammation. PloS one. 2012; 7:e29371.
9. Sage A P, Tsiantoulas D, Baker L, Harrison J, Masters L, Murphy D, Loinard C, Binder C J and Mallat Z. BAFF receptor deficiency reduces the development of atherosclerosis in mice—brief report. Arteriosclerosis, thrombosis, and vascular biology. 2012; 32:1573-6.
10. Tanigaki K, Sundgren N, Khera A, Vongpatanasin W, Mineo C and Shaul P W. Fcgamma receptors and ligands and cardiovascular disease. Circulation research. 2015; 116:368-84.
11. Liu Y, Xu L, Opalka N, Kappler J, Shu H B and Zhang G. Crystal structure of STALL-1 reveals a virus-like assembly of TNF family ligands. Cell. 2002; 108:383-94.
12. Bossen C, Cachero T G, Tardivel A, Ingold K, Willen L, Dobles M, Scott M L, Maquelin A, Belnoue E, Siegrist C A, Chewier S, Acha-Orbea H, Leung H, Mackay F, Tschopp J and Schneider P. TACI, unlike BAFF-R, is solely activated by oligomeric BAFF and APRIL to support survival of activated B cells and plasmablasts. Blood. 2008; 111:1004-12.
13. Navarra S V, Guzman R M, Gallacher A E, Hall S, Levy R A, Jimenez R E, Li E K; Thomas M, Kim H Y, Leon M G, Tanasescu C, Nasonov E, Lan J L, Pineda L, Zhong Z J, Freimuth V V, Petri M A and Group B-S, Efficacy and safety of belimumab in patients with active systemic lupus erythematosus: a randomised, placebo-controlled, phase 3 trial. Lancet. 2011; 377:721-31.
14. Ait-Oufella H, Herbin O, Bouaziz J D, Binder C J, Uyttenhove C, Laurans L, Taleb S, Van Vre E, Esposito B, Vilar J, Sirvent J, Van Snick J, Tedgui A, Tedder T F and Mallat Z. B cell depletion reduces the development of atherosclerosis in mice. The Journal of experimental medicine. 2010; 207:1579-87.
15. Eliason J L, Hannawa K K, Ailawadi G, Sinha I, Ford J W, Deogracias M P, Roelofs K J, Woodrum D T, Ennis T L, Henke P K, Stanley J C, Thompson R W and Upchurch G R, Jr, Neutrophil depletion inhibits experimental abdominal aortic aneurysm formation. Circulation. 2005; 112:232-40.
16. Puga I, Cols M, Barra C M, He B, Cassis L, Gentile M, Comerma L, Chorny A, Shan M, Xu W, Magri G, Knowles D M, Tarn W, Chiu A, Bussel J B, Serrano S, Lorente J A, Bellosillo B, Lloreta J, Juanpere N, Alameda F, Baro T, de Heredia C D, Toran N, Catala A, Torrebadell M, Fortuny C, Cusi V, Carreras C, Diaz G A, Blander J M, Farber C M, Silvestri G, Cunningham-Rundles C, Calvillo M, Dufour C, Notarangelo L D, Lougaris V, Plebani A, Casanova J L, Ganal S C, Diefenbach A, Arostegui J I, Juan M, Yague J, Mahlaoui N, Donadieu J, Chen K and Cerutti A. B cell-helper neutrophils stimulate the diversification and production of immunoglobulin in the marginal zone of the spleen. Nat Immunol. 2011; 13:170-80,
17. Parsa R, Lund H, Georgoudaki A M, Zhang X M, Ortlieb Guerreiro-Cacais A, Grommisch D, Warnecke A, Croxford A L, Jagodic M, Becher B, Karlsson M C and Harris R A. BAFF-secreting neutrophils drive plasma cell responses during emergency granulopoiesis. The Journal of experimental medicine. 2016; 213:1537-53,
18. Bossen C, Tardivel A, Willen L, Fletcher C A, Perroud M, Beermann F, Rolink A G, Scott M L, Mackay F and Schneider P. Mutation of the BAFF Turin cleavage site impairs B-cell homeostasis and antibody responses. Eur J Immunol. 2011; 41:787-97,
19. Cachero T G, Schwartz I M, Qian F, Day E S, Bossen C, Ingold K, Tardivel A, Krushinskie D, Eldredge J, Silvian L, Lugovskoy A, Farrington G K, Strauch K, Schneider P and Whitty A. Formation of virus-like clusters is an intrinsic property of the tumor necrosis factor family member BAFF (B cell activating factor). Biochemistry. 2006; 45:2006-13.
20. Castigli E, Wilson S A, Scott 5, Dedeoglu F, Xu 5; Lam K P, Bran R J, Jabara H and Geha R S. TACT and BAFF-R mediate isotype switching in B cells. The Journal of experimental medicine. 2005; 201:35-9,
21. Sasaki Y, Casola S, Kutok J L, Rajewsky K and Schmidt-Supprian M. TNF family member B cell-activating factor (BAFF) receptor-dependent and -independent roles for BAFF in B cell physiology. Journal of immunology. 2004; 173:2245-52.
22. Schiemann B, Gommerman J L, Vora K, Cachero T G, Shulga-Morskaya S, Dobles M, Frew E and Scott M L. An essential role for BAFF in the normal development of B cells through a BCMA-independent pathway. Science. 2001; 293:2111-4.
23. Shen L, Chng M H, Alonso M N, Yuan R, Winer D A and Engleman E G, B-1a lymphocytes attenuate insulin resistance. Diabetes. 2015; 64:593-603.
24. Kowalczyk-Quintas C, Schuepbach-Mallepell S, Vigolo M, Willen L, Tardivel A, Smulski C R, Zheng T S, Gommerman J, Hess H, Gottenberg J E, Mackay F, Donze O and Schneider P. Antibodies That Block or Activate Mouse B Cell Activating Factor of the Tumor Necrosis Factor (TNF) Family (BAFF), Respectively, Induce B Cell Depletion or B Cell Hyperplasia. J Biol Chem. 2016; 291:19826-34.
25. Yu M, Chen Y, He Y, Podd A, Fu G, Wright JA, Kleiman E, Khan W N, Wen R and Wang D. Critical role of B cell lymphoma 10 in BAFF-regulated NF-kappaB activation and survival of anergic B cells. Journal of immunology. 2012; 189:5185-93.
26. Kanno Y, Sakurai D, Hase H, Kojima H and Kobata T. TACI induces cIAP1-mediated ubiquitination of NIK by TRAF2 and TANK to limit non-canonical NF-kappaB signaling. J Recept Signal Transduct Res. 2010; 30:121-32.
27. Caro-Maldonado A, Wang R, Nichols AG, Kuraoka M, Milasta S, Sun L D Gavin A L, Abel E D, Kelsoe G, Green DR and Rathmell J C. Metabolic reprogramming is required for antibody production that is suppressed in anergic but exaggerated in chronically BAFF-exposed B cells. Journal of immunology. 2014; 192:3626-36.
28. Lam W Y, Becker A M, Kennerly K M, Wong R, Curtis J D, Llufrio E M, McCommis K S, Fahrmann J, Pizzato H A, Nunley R M, Lee J, Wolfgang M J, Patti G J, Finck B N, Pearce E L and Bhattacharya D. Mitochondrial Pyruvate Import Promotes Long-Term Survival of Antibody-Secreting Plasma Cells. Immunity. 2016; 45:60-73.
29. Furusho A, Aoki H, Ohno-Urabe S, Nishihara M, Hirakata S, Nishida N, Ito S, Hayashi M, Imaizumi T, Hiromatsu S, Akashi H, Tanaka H and Fukumoto Y. Involvement of B Cells, Immunoglobulins, and Syk in the Pathogenesis of Abdominal Aortic Aneurysm, Journal of the American Heart Association. 2018; 7.
30. Kelly J A, Griffin M E, Fava R A, Wood S G, Bessette K A, Miller E R, Huber S A, Binder C J, Witztum J L and Morganelli P M. Inhibition of arterial lesion progression in CD16-deficient mice: evidence for altered immunity and the role of IL-10. Cardiovasc Res. 2010; 85:224-31,
31. Huang Y, Yin H, Wang J, Liu O, Wu C and Chen K. Aberrant expression of FcgammaRIIIA (CD16) contributes to the development of atherosclerosis. Gene. 2012; 498:91-5.
32. Huang Y, Yin H, Wang J, Ma X, Zhang Y and Chen K. The significant increase of FcgammaRIIIA (CD16), a sensitive marker, in patients with coronary heart disease. Gene. 2012; 504:284-7.
33. Blom A B, Radstake T R, Holthuysen A E, Sloetjes A W, Pesman G J, Sweep F G, van de Loo F A, Joosten L A, Barrera P, van Lent P L and van den Berg W B. Increased expression of Fcgamma receptors H and HI on macrophages of rheumatoid arthritis patients results in higher production of tumor necrosis factor alpha and matrix metalloproteinase. Arthritis Rheum. 2003; 48:1002-14.
34. Nicoletti A M, Kenny C H, Khalil A M, Pan O, Ralph K L, Ritchie J, Venkataramani S, Presky D H, DeWire S M and Brodeur S R. Unexpected Potency Differences between B-Cell-Activating Factor (BAFF) Antagonist Antibodies against Various Forms of BAFF: Trimer, 60-Mer, and Membrane-Bound. J Pharmacal Exp Ther. 2016; 359:37-44.
35. Vigolo M, Chambers M G, Willen L, Chevalley D, Maskos K, Lammens A, Tardivel A, Das D, Kowalczyk-Quintas C, Schuepbach-Mallepell S, Smulski C R, Eslami M, Rolink A, Hummler E, Samy E, Fomekong Nanfack Y, Mackay F, Liao M, Hess H, Jiang X and Schneider P. A loop region of BAFF controls B cell survival and regulates recognition by different inhibitors. Nat Commun. 2018; 9:1199.
36. Shin W, Lee H T, Lim H, Lee S H, Son J Y, Lee J U, Yoo K Y, Ryu S E, Rhie J, Lee J Y and Heft Y S. BAFF-neutralizing interaction of belimumab related to its therapeutic efficacy for treating systemic lupus erythematosus. Nat Commun. 2018; 9:1200.
37. Guirguis-Blake J M, Beil T L, Seager C A and Whitlock E R Ultrasonography screening for abdominal aortic aneurysms: a systematic evidence review for the U.S. Preventive Services Task Force. Annals of internal medicine. 2014; 160:321-9.
38. Bown M J, Sutton A J, Bell P R and Sayers R D. A meta-analysis of 50 years of ruptured abdominal aortic aneurysm repair. The British journal of surgery. 2002; 89:714-30.
39. Baxter B T, Matsumura J, Curci J, McBride R, Blackwelder W C, Liu X, Larson L, Terrin M L and Investigators N T. Non-invasive Treatment of Abdominal Aortic Aneurysm Clinical Trial (N-TA(3)CT): Design of a Phase Hb, placebo-controlled, double-blind; randomized clinical trial of doxycycline for the reduction of growth of small abdominal aortic aneurysm. Contemp Clin Trials. 2016; 48:91-8.
40. Meijer C A; Stijnen T, Wasser M N, Hamming J F, van Bockel J H, Lindeman J H and Pharmaceutical Aneurysm Stabilisation Trial Study G. Doxycycline for stabilization of abdominal aortic aneurysms: a randomized trial. Ann Intern Med. 2013; 159:815-23.
41. Pearce E L, Poffenberger M C; Chang C H and Jones R G. Fueling immunity: insights into metabolism and lymphocyte function. Science. 2013; 342:1242454.
42. Pearce E L and Pearce E J. Metabolic pathways in immune cell activation and quiescence. Immunity. 2013; 38:633-43.
43. O'Sullivan D and Pearce E L. Targeting T cell metabolism for therapy. Trends in immunology. 2015; 36:71-80.
44. Buck M D, O'Sullivan D and Pearce E L. T cell metabolism drives immunity. The Journal of experimental medicine. 2015; 212:1345-60.
45. Mockler M B, Conroy M J and Lysaght J. Targeting T cell immunometabolism for cancer immunotherapy; understanding the impact of the tumor microenvironment. Frontiers in oncology. 2014; 4:107.
46. Kurosaki T. Regulation of B-cell signal transduction by adaptor proteins. Nat Rev Immunol. 2002; 2:354-63.
47. Allende M L, Tuymetova G, Lee B G, Bonifacino E, Wu Y P and Proia R L. S1P1 receptor directs the release of immature B cells from bone marrow into blood, The Journal of experimental medicine. 2010; 207:1113-24.
48. Coope H J, Atkinson P G, Huhse B, Belich M, Janzen J, Holman M J, Klaus G G, Johnston L H and Ley S C. CD40 regulates the processing of NE-kappaB2 p100 to p52. EMBO J. 2002; 21:5375-85.
49. Meher A K, Bal N C, Chary KV and Arora A. Mycobacterium tuberculosis H37Rv ESAT-6-CFP-10 complex formation confers thermodynamic and biochemical stability. FEBS J. 2006; 273:1445-62.
50. Bal N C, Agrawal H, Meher A K and Arora A, Characterization of peptidyl-tRNA hydrolase encoded by open reading frame Rv1014c of Mycobacterium tuberculosis H37Rv, Biol Chem. 2007; 388:467-79.
51. Bal N C, Agrawal H, Meher A K, Pulavarti SV, Jain A, Kelly G, Frenkiel T A, Pastore A and Arora A. NMR assignment of peptidyl-tRNA hydrolase from Mycobacterium tuberculosis H37Rv. J Biomol NMR. 2006; 36 Suppl 1:53.
52. Meher A K, Lelia R K, Sharma C and Arora A. Analysis of complex formation and immune response of CFP-10 and ESAT-6 mutants. Vaccine. 2007; 25:6098-106.
53. Carrigan S O, Weppler A L, Issekutz A C and Stadnyk A W. Neutrophil differentiated HL-60 cells model Mac-1 (CD11b/CD18)-independent neutrophil transepithelial migration. Immunology. 2005; 115:108-17.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A composition for inhibiting a soluble B cell activating factor (BAFF) biological activity, the composition comprising a reagent that interacts with a BAFF polypeptide or BAFF gene product and a carrier, whereby inhibition of BAFF multimerization substantially without depletion of mature B cells is accomplished.

2. The composition of claim 1, wherein the composition comprises an anti-BAFF antibody that binds to a BAFF polypeptide, a DE loop mimetic peptide, and/or a nucleic acid that binds to a BAFF gene product.

3. The composition of claim 1 or claim 2, wherein the anti-BAFF antibody binds to an epitope present within a DE loop of a BAFF polypeptide and/or comprising a subsequence thereof, wherein depletion of mature B cells is substantially avoided.

4. The composition of any one of the preceding claims, wherein the BAFF biological activity is selected from the group consisting of BAFF multimerization, binding of BAFF to a cognate receptor, inducing signal transduction mediated by a cognate receptor, and modulating growth of an abdominal aortic aneurysm (AAA).

5. The composition of any one of the preceding claims, wherein BAFF multimerization comprises multimerization of a trimer form of BAFF to a 60mer form of BAFF.

6. The composition of any one of the preceding claims, wherein the cognate receptor is selected from the group consisting of BAFF receptor (BR3), CAML interactor (TACI), and B-cell maturation antigen (BCMA).

7. A pharmaceutical composition comprising the composition of any of the preceding claims, optionally wherein the pharmaceutical composition is pharmaceutically acceptable for use in a human.

8. An anti-BAFF antibody formulation, the anti-BAFF antibody formulation prepared by immunizing a mammal with an antigen comprising an BAFF peptide or polypeptide, optionally wherein the antigen comprises, consists essentially of, or consists of the amino acid sequence KVHVFGDELS (SEQ ID NO: 1), and further optionally wherein the amino acid sequence KVHVFGDELS (SEQ ID NO: 1) is conjugated to a carrier.

9. The anti-BAFF antibody formulation of claim 8, wherein the mammal is selected from the group consisting of a rabbit and a mouse.

10. The anti-BAFF antibody formulation of claim 8 or of claim 9, wherein the antigen comprises, consists essentially of, or consists of amino acid sequence KVHVFGDELSLVT (SEQ ID NO: 2) or amino acid sequence KVHVFGDELS (SEQ ID NO: 1), optionally conjugated to keyhole limpet hemocyanin (KLH) via an N-terminal or a C-terminal cysteine addition.

11. The anti-BAFF antibody formulation of any one of claims 8-10, wherein the mammal is a mouse and the anti-BAFF antibody is a monoclonal antibody.

12. The anti-BAFF antibody formulation of any one of claims 8-11, wherein the formulation inhibits BAFF multimerization substantially without depletion of mature B cells.

13. A method for inhibiting a biological activity of a BAFF gene product, the method comprising contacting the BAFF gene product with an effective amount of an inhibitor of BAFF, wherein the inhibitor comprises a reagent that interacts with a BAFF polypeptide or BAFF-encoding nucleic acid sequence, whereby inhibition of BAFF multimerization substantially without depletion of mature B cells is accomplished.

14. The method of claim 13, wherein the inhibitor of BAFF is selected from the group consisting of an anti-BAFF antibody, a DE loop mimetic peptide, and an anti-BAFF inhibitory nucleic acid.

15. The method of claim 14, wherein the anti-BAFF antibody binds to an epitope present within a DE loop of a BAFF polypeptide and/or comprising a subsequence thereof, to thereby inhibit BAFF multimerization, inhibit binding of BAFF to a cognate receptor, and/or inhibit signal transduction mediated by a cognate receptor.

16. The method of any one of claims 13-15, wherein the biological activity of the BAFF gene product is associated with growth of an abdominal aortic aneurysm (AAA).

17. A method for inhibiting growth of an abdominal aortic aneurysm (AAA), the method comprising administering to a subject in need thereof an effective amount of a composition for inhibiting a soluble B cell activating factor (BAFF) biological activity.

18. The method of claim 17, wherein the composition for inhibiting a soluble B cell activating factor (BAFF) biological activity comprises a reagent that interacts with a BAFF polypeptide or BAFF-encoding nucleic acid sequence and a carrier.

19. The method of claim 17 or claim 18, wherein the composition inhibits BAFF multimerization substantially without depletion of mature B cells.

20. The method of any one of claims 17-19, wherein the composition comprises an anti-BAFF antibody that binds to a BAFF polypeptide, a DE loop mimetic peptide, and/or a nucleic acid that binds to a BAFF gene product.

21. The method of any one of claims 17-20, wherein the anti-BAFF antibody binds to an epitope present within a DE loop of a BAFF polypeptide and/or comprising a subsequence thereof.

22. The method of any one of claims 17-21, wherein the BAFF biological activity is selected from the group consisting of BAFF multimerization, binding of BAFF to a cognate receptor, inducing signal transduction mediated by a cognate receptor, and modulating growth of an abdominal aortic aneurysm (AAA).

23. The method of any one of claims 19-22, wherein BAFF multimerization comprises multimerization of a trimer form of BAFF to a 60mer form of BAFF.

24. The method of claim 22, wherein the cognate receptor is selected from the group consisting of BAFF receptor (BR3), CAML interactor (TACO, and B-cell maturation antigen (BCMA).

25. The method of any one of claims 17-24, wherein the composition comprises a pharmaceutically acceptable carrier, optionally wherein the carrier is pharmaceutically acceptable for use in a human.

26. A method of treating a B cell-related condition, the method comprising administering to a subject in need thereof an effective amount of a composition of any one of claims 1-7.

27. The method of claim 17, wherein the B cell-related condition comprises a condition selected from the group consisting of an abdominal aortic aneurysm (AAA), a cardiovascular disease, lupus, type 1 diabetes, type 2 diabetes, and a B-cell related cancer.


SEQUENCE LISTING (14 sequences)

SEQ ID NO: 1 - protein, 10 aa, Homo sapiens
KVHVFGDELS

SEQ ID NO: 2 - protein, 13 aa, Homo sapiens
KVHVFGDELSLVT

SEQ ID NO: 3 - protein, 20 aa, Homo sapiens
LIQRKKVHVFGDELSLVTLF

SEQ ID NO: 4 - protein, 18 aa, Homo sapiens
IQRKKVHVFGDELSLVTL

SEQ ID NO: 5 - protein, 12 aa, Homo sapiens
KKVHVFGDELSL

SEQ ID NO: 6 - protein, 15 aa, Homo sapiens
LIQRKKVHVFGDELS

SEQ ID NO: 7 - protein, 16 aa, Homo sapiens
LIQRKKVHVFGDELSL

SEQ ID NO: 8 - protein, 16 aa, Homo sapiens
IQRKKVHVFGDELSLV

SEQ ID NO: 9 - protein, 16 aa, Homo sapiens
QRKKVHVFGDELSLVT

SEQ ID NO: 10 - protein, 16 aa, Homo sapiens
RKKVHVFGDELSLVTL

SEQ ID NO: 11 - protein, 12 aa, Homo sapiens
LIQRKKVHVFGD

SEQ ID NO: 12 - protein, 18 aa, Artificial Sequence
  Artificially synthesized peptide that can be cyclicized
IQRKKVHVFGDELSLVTL

SEQ ID NO: 13 - protein, 8 aa, Artificial Sequence
  Artificially synthesized peptide that can be cyclicized
VHVFGDEL

SEQ ID NO: 14 - protein, 14 aa, Artificial Sequence
  Artificially synthesized peptide DE4akm
RKKVHVFGDELSLV